(19) United States
(12) Patent Application Publication
(10) Pub. No.: US 2026/0226494 A1
(43) Pub. Date: Aug. 6, 2026

(54) Transgenic Soybean Event XP-2 and Detection Method Thereof

(71) Applicant: HANGZHOU RUIFENG BIOSCIENCE CO., LTD., Hangzhou, Zhejiang (CN)

(72) Inventors: Ting ZHENG, Hangzhou, Zhejiang (CN); Pengfei WANG, Hangzhou, Zhejiang (CN); Chao XU, Hangzhou, Zhejiang (CN); Haiyan LIN, Hangzhou, Zhejiang (CN); Xuezhen XU, Hangzhou, Zhejiang (CN); Yuanyuan JIANG, Hangzhou, Zhejiang (CN); Xiaoxing YU, Hangzhou, Zhejiang (CN); Mengzhen TANG, Hangzhou, Zhejiang (CN); Jing LI, Hangzhou, Zhejiang (CN); Hongying TONG, Hangzhou, Zhejiang (CN); Hong JIANG, Hangzhou, Zhejiang (CN); Chun JIANG, Hangzhou, Zhejiang (CN); Chaoyang LIN, Hangzhou, Zhejiang (CN); Zhicheng SHEN, Hangzhou, Zhejiang (CN)

(21) Appl. No.: 19/151,879

(22) PCT Filed: Jul. 23, 2024
(86) PCT No.: PCT/CN2024/106956

§ 371 Date: Jul. 29, 2025

(30) Foreign Application Priority Data
Oct. 23, 2023 (CN) 202311375014.7

(51) Int. Cl. C12N 15/82; A01N 43/66; A01N 57/20; A01P 13/02; C12Q 1/6895

(52) U.S. Cl. CPC C12N 15/8275; A01N 43/66; A01N 57/20; A01P 13/02; C12N 15/8278; C12Q 1/6895; C12Q 2600/13; C12Q 2600/156

(57) ABSTRACT

A transgenic soybean event XP-2 and a detection method thereof. The transgenic soybean event XP-2 utilizes the nucleotide sequence shown in SEQ ID NO:27 as the left flanking region of the exogenous genes and the nucleotide sequence shown in SEQ ID NO:28 as the right flanking region of the exogenous genes. The transgenic soybean event XP-2 co-localizes genes encoding glyphosate tolerance and flazasulfuron tolerance traits on the same DNA segment, and these genes are integrated into a single locus within the genome of the transgenic soybean event XP-2. This configuration provides enhanced breeding efficiency and enables the use of molecular markers to track the transgenic insert in breeding populations and their progeny. The specific nucleic acid sequences for detecting soybean plants allow for the unique identification of transgenic soybean event XP-2.

### REFERENCE TO AN ELECTRONIC SEQUENCE LISTING

The content of the electronic sequence listing (Sequence Listing.xml; Size: 57,344 bytes; and Date of Creation: Jul. 29, 2025) is herein incorporated by reference in its entirety.

### TECHNICAL FIELD

This invention relates to a herbicide-tolerant transgenic soybean event and its detection method, particularly relates to the transgenic soybean event XP-2 tolerant to flazasulfuron and glyphosate herbicides, nucleic acid sequence for detecting the presence of the specific transgenic soybean event XP-2 in biological samples and its detection method.

### BACKGROUND TECHNOLOGY

Soybean (Glycine max) is one of the major crops in many regions worldwide, with herbicide tolerance being one of its important agronomic traits. Biotechnology has been applied to soybeans to improve their agronomic traits and quality. Through transgenic methods, genes such as EPSPS for glyphosate tolerance and P450 for flazasulfuron tolerance can be expressed in soybean plants. For example, the cp4 epsps gene derived from Agrobacterium tumefaciens sp strain CP4 was introduced into crops through Agrobacterium-mediated transformation, the expression of 5-enolpyruvylshikimate-3-phosphate synthase (EPSPS) in these crops conferred tolerance to the herbicide glyphosate. China has developed several transgenic crops with the cp4 epsps gene, such as corn (Zea mays), rapeseed (Brassica napus), sugar beet (Beta vulgaris), and alfalfa (Medicago sativa). However, prolonged use of a single herbicide often leads to the emergence of a large number of herbicide-tolerant weeds. In the Americas, the promotion of glyphosate-tolerant crops for over two decades has already resulted in the production of numerous glyphosate-tolerant weeds, making it difficult to effectively control weeds in the field with glyphosate alone. Developing transgenic crops that are tolerant to two or more herbicides can provide diversified options for weed control and effectively delay the emergence of herbicide-tolerant weeds.

Cytochrome P450 is a superfamily of genes with numerous members. Research has shown that the P450 enzyme system is involved in the metabolism and detoxification of various types of herbicides and plays a crucial role in the development of herbicide tolerance. A P450 gene N-Z1 in CYrnodon dactylon (U.S. Pat. No. 9,657,303, Canadian Patent: CA2818581C, and Brazilian Patent: BR112013012678B1) encodes a protein that confers tolerance to multiple herbicides in crops.

It is known that the expression of transgenic genes is influenced by their chromosomal location. Due to their different locations, there can be significant differences in the expression levels, spatial and temporal patterns of exogenous genes, which in turn leads to varying impacts on the agronomic traits of plants. Different transformation events derived from the same exogenous gene often exhibit significant differences in traits: therefore, each independent transformation event has a distinct impact on the recipient plant. Obtaining plant transformation events that enable efficient expression of exogenous genes without compromising the plant's inherent agronomic traits holds significant value in the development of new transgenic crop varieties.

It would be beneficial to have methods for detecting the presence of specific events to determine whether sexually hybridized offspring contain the gene of interest. This has important value for the hybrid breeding, production applications, commercial registration, and regulatory compliance of transgenic crops. Providing information on the integration sites of exogenous genes in transformation events allows for the detection of the presence of transformation events in plants using existing polynucleotide-based detection methods. Conventional methods for detecting polynucleotides and proteins can identify whether an organism is transgenic but cannot effectively distinguish between different transformation events, especially those generated using the same gene or transformation vector. Therefore, only by detecting the inserted gene and its flanking sequences can the presence of a target transgenic event be accurately determined.

### SUMMARY OF THE INVENTION

The purpose of the present invention is to provide a transgenic soybean event XP-2 and a detection method thereof, the event simultaneously expresses two proteins, CdP450 and cp4 epsps, conferring high tolerance to flazasulfuron and glyphosate, and has genetic stability and no adverse effects on agronomic traits; additionally, the present invention provides a method for detecting the transgenic soybean event XP-2, which can effectively identify the specific transgenic soybean event XP-2, solving the problem of transformant identity recognition, and be used for molecular detection of transgenic soybean XP-2 during breeding, cultivation, and regulatory processes.

Technical solutions adopted by the present invention are as follows:

The first aspect of the present invention provides a nucleic acid sequence of the transgenic soybean event XP-2, which comprises one or more selected from the group consisting of SEQ ID NO:1, SEQ ID NO:2, and their complementary sequences.

Furthermore, the nucleic acid sequence comprises one or more selected from the group consisting of SEQ ID NO:1-10 and their complementary sequences.

Still further, the nucleic acid sequence is derived from a plant, seed, or cell comprising the transgenic soybean event XP-2, and a representative sample of seeds comprising the transgenic soybean event XP-2 is deposited under Accession Number CCTCC NO: P202329.

The transgenic soybean event XP-2 uses the nucleotide sequence shown in SEQ ID NO:27 as the left flanking region of exogenous genes and the nucleotide sequence shown in SEQ ID NO:28 as the right flanking region of the exogenous genes. The exogenous genes include the flazasulfuron-tolerant gene CdP450 and the glyphosate-tolerant gene cp4 epsps.

The transgenic soybean event XP-2 of the present invention is obtained by introducing an exogenous T-DNA sequence containing a flazasulfiuron-tolerant gene expression cassette and a glyphosate-tolerant gene expression cassette into the soybean genome using an Agrobacterium-mediated soybean cotyledon node transformation method, obtaining soybean transgenic populations by regenerating soybean cells containing exogenous genes, and screening the soybean transformation event that meets production needs using molecular biology and bioassay methods. Progeny of the transgenic soybean event XP-2 of the invention are obtained through methods such as hybridization, and any soybean event and seed with the exogenous T-DNA flank sequences being SEQ ID NO: 27 and SEQ ID NO: 28 in the genomes of progeny should be considered as an aspect of the invention.

Furthermore, the T-DNA (FIG. 1) comprising the exogenous genes mentioned in the invention comprises two linked plant expression cassettes, namely the flazasulfuron-tolerant gene expression cassette and glyphosate-tolerant gene expression cassette, wherein regulatory genetic elements are necessary for expressing flazasulfuron-tolerant CdP450 and glyphosate-tolerant cp4 epsps in soybean plant cells. The flazasulfuron-tolerant gene expression cassette encodes a flazasulfuron-tolerant cytochrome P450 oxidase and expresses the CdP450 protein; the flazasulfuron-tolerant CdP450 gene expression cassette is composed of the pCsVMiV promoter, the flazasulfuron-tolerant gene CdP450, and the CaMV35S terminator, wherein the pCsVMV promoter is a constitutive promoter derived from the figwort mosaic virus and can drive the expression of the target gene in all plant tissues, and the terminator is the 35S terminator derived from the tobacco mosaic virus. The glyphosate-tolerant gene expression cassette encodes a key enzyme with glyphosate tolerance characteristics in the synthetic paihway of aroniaic amino acids in plants and expresses the cp4 epsps protein; the glyphosate-tolerant cp4 epsps gene expression cassette is composed of the Atubi promoter, the rice EPSPS signal peptide, the cp4 epsps gene, and the 35S terminator; wherein the 35S promoter is a constitutive promoter derived from the cauliflower mosaic virus and can drive the expression of the target gene in all plant tissues, the rice EPSPS signal peptide is derived from rice, cp4 epsps is derived from Agrobacterium tumefaciens strain CP4, and the 35S terminator is derived from the cauliflower mosaic virus.

Furthermore, the nucleotide sequence of the T-DNA comprising the exogenous genes is preferably as shown in SEQ ID NO:9.

The transgenic soybean event XP-2 of the invention is a DNA construct containing the exogenous T-DNA, including a left flanking sequence, exogenous genes, and a right flanking sequence, with a nucleotide sequence as shown in SEQ ID NO:10.

The present invention also provides a recombinant vector or recombinant cell containing the transgenic soybean event XP-2, which contains the T-DNA insertion sequence.

The second aspect of the present invention also relates to a specific nucleotide sequence for detecting the transgenic soybean event XP-2, the specific nucleotide sequence has a linker sequence between the left or right flanking region and the exogenous genes, and the linker sequence comprises the nucleotide sequence of the exogenous DNA inserted into the soybean genome and the nucleotide sequence of DNA in the left or right flanking region of the soybean cell genome.

Furthermore, the specific nucleotide sequence has at least 11 consecutive nucleotides of one of the nucleotide sequences shown in SEQ ID NO: 1, SEQ ID NO:3, SEQ ID NO:5, SEQ ID NO:7, or their complementary sequences, for detecting the insertion of the exogenous gene at the 5′ end. When an amplicon contains SEQ ID NO:1, SEQ ID NO:3, SEQ ID NO:5, SEQ ID NO:7, or their complementary sequences, the presence of the transgenic soybean XP-2 can be identified.

Furthermore, the specific nucleotide sequence has at least 11 consecutive nucleotides of one of the nucleotide sequences shown in SEQ ID NO:2, SEQ ID NO:4, SEQ ID NO:6, SEQ ID NO:8, or their complementary sequences, for detecting the insertion of the exogenous gene at the 3′ end. When an amplicon contains SEQ ID NO:2, SEQ ID NO:4, SEQ ID NO:6, SEQ ID NO:8, or their complementary sequences, the presence of the transgenic soybean XP-2 can be identified.

Furthermore, the specific nucleotide sequence has the nucleotide sequence shown in SEQ ID NO:10 or its complementary sequence. When an amplicon contains SEQ ID NO:10 or its complementary sequence, the presence of the transgenic soybean XP-2 can be identified.

The present invention provides a consecutive nucleotide sequence specific to the transgenic soybean event XP-2, this consecutive nucleotide sequence can be used to characterize the transgenic soybean event XP-2, thereby enabling detection of the presence of transgenic soybean event XP-2 in a sample. Specifically, the presence of at least 11 consecutive nucleotides from one or more of the nucleic acid molecules shown in SEQ ID NO: 1-10 in the sample indicates the presence of the transgenic soybean event XP-2 in said sample.

The third aspect of the present invention provides a primer pair for detecting said transgenic soybean event XP-2, comprising a first primer and a second primer different from the first primer, and the first primer and the second primer each comprise a portion of SEQ ID NO:10 or its complementary sequence for detecting the transgenic soybean event XP-2 in a sample.

Furthermore, the first primer is one selected from SEQ ID NO:22 and SEQ ID NO:25; and the second primer is one selected from SEQ ID NO:23 and SEQ ID NO:26.

A DNA probe for detecting the transgenic soybean event XP-2, said DNA probe comprises a portion of SEQ ID NO:10 or its complementary sequence, wherein the probe hybridizes under stringent hybridization conditions to DNA containing any of SEQ ID NO:1-10 or their complementary sequences, but does not hybridize to DNA lacking these sequences.

Furthermore, said DNA probe is as shown in SEQ ID NO:24, and said probe is labeled with at least one fluorescent group, preferably 6FAM™ (6-Carboxyfluorescein).

The fourth aspect of the present invention provides a method for detecting the presence of DNA comprising the transgenic soybean event XP-2 in a sample using said primer pair and DNA probe, comprising: (1) contacting the sample to be tested with the DNA probe or primer pair in a nucleic acid amplification reaction mixture; (2) performing nucleic acid amplification; and (3) detecting the presence of DNA amplicons, wherein said DNA amplicons comprise at least 11 consecutive nucleotides of one of SEQ ID NO:1 to SEQ ID NO:10 or their complementary sequences, preferably at least 11 consecutive nucleotides of SEQ ID NO:1 or its complementary sequence, and/or at least 1 consecutive nucleotides of SEQ ID NO:2 or its complementary sequence.

The fifth aspect of the present invention further provides a method for cultivating herbicide-tolerant soybean plants containing said transgenic soybean event XP-2, comprising: planting soybean seeds containing a specific nucleotide sequence, spraying a herbicide, and harvesting soybeans having significantly enhanced herbicide tolerance compared to other soybean plants lacking the specific nucleic acid sequence; said specific nucleotide sequence is selected from one of SEQ ID NO: 1, SEQ ID NO:2, SEQ ID NO:3, SEQ ID NO:4, SEQ ID NO:5, SEQ ID NO:6, SEQ ID NO:7, SEQ ID NO: 8. SEQ ID NO:9, SEQ ID NO: 10, or their complementary sequences; and said herbicide include glyphosate or flazasulfuron.

The sixth aspect of the present invention further provides a method for controlling field weeds in the cultivation of soybean plants containing said transgenic soybean event XP-2, comprising: planting transgenic soybean plants containing a specific nucleotide sequence, and spraying with effective doses of glyphosate and/or flazasulfuron herbicides to control the weeds; the genome of said transgenic soybean comprises the specific nucleotide sequence from the transgenic soybean event XP-2, which include one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or their complementary sequences.

The seventh aspect of the present invention further provides a method for obtaining flazasulfuron-tolerant and/or glyphosate-tolerant soybean plants based on said transgenic soybean event XP-2, comprising: hybridizing the soybean plant containing a specific nucleotide sequence with another soybean plant to produce progeny plants; harvesting plants with significantly enhanced tolerance to herbicides compared to plants lacking the specific nucleotide sequence; said specific nucleotide sequence is derived from the transgenic soybean event XP-2 and comprises one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, or their complementary sequences. Progeny of the transgenic soybean event XP-2 include plants, or seeds, or plant parts and seeds, and the plant parts comprise but not limited to: pollen, ovules, petals, stems, leaves, seeds, pods, and meristematic tissues.

The eighth aspect of the present invention provides a transgenic plant cell derived from the transgenic soybean event XP-2, obtained by introducing a specific regional nucleic acid sequence of said transgenic soybean event XP-2 into the plant genome, wherein said specific regional nucleic acid sequence comprise one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or their complementary sequences.

The ninth aspect of the present invention provides a soybean commodity or agricultural product produced from the transgenic soybean event XP-2, containing a detectable DNA molecule originating from the specific nucleotide sequence of the transgenic soybean event XP-2, which is selected from one of SEQ ID NO:1-10 or their complementary sequences; said soybean commodity or agricultural product includes: soybean oil, soybean protein, soybean meal, soybean flour, soybean flakes, soybean hulls, soymilk, soy cheese, soy wine, animal feed containing soybeans, paper containing soybeans, cheese containing soybeans, soybean biomass, and fuel products produced using soybean plants and soybean plant parts.

The tenth aspect of the present invention provides a soybean seed XP-2 (Glycine max μL. Merr. XP-2) containing the transgenic soybean event XP-2, deposited at the China Center for Type Culture Collection under accession number CCTCC NO: P202329 on Jul. 9, 2023, with deposit address: Wuhan University, Wuhan, China, Zip Code 430072.

The term “soybean” as used herein refers to Glycine max and includes all plant varieties that can be used for breeding soybean plants containing the transgenic soybean event XP-2, encompassing wild soybean species and those plants of the genus Glycine that are permissible for interspecies breeding. The term “includes” as used herein means “includes but is not limited to”.

The term “Flanking DNA” as used herein may comprise genomic DNA naturally occurring in organisms such as plants or exogenous (heterologous) DNA introduced through transformation processes, such as fragments associated with the transformation events. Therefore, Flanking DNA may include combinations of both native and exogenous DNA. In the present invention, the terms “flanking region,” “flanking sequence,” “genomic border region,” or “genomic border sequence” refer to sequences having at least 3, 5, 10, 11, 14, 15, 20, 50, 100, 200, 300, 400, 1000, 1500, 2000, 2500, or 5000 base pairs or longer, which are located directly upstream or downstream of and adjacent to the original exogenous inserted DNA molecule. When the flanking region is located downstream, it may also be referred to as “left border flanking,” “3′ flanking,” “3′ genomic border region,” or “genomic 3′ border sequence,” etc. When the flanking region is located upstream, it may also be referred to as “right border flanking,” “5′ flanking,” “5′ genomic border region,” or “genomic 5′ border sequence,” etc.

The transformation process causing random integration of exogenous DNA results in transformation events containing different flanking regions, the different flanking regions are specifically contained by each transformation event. When recombinant DNA is introduced into plants through traditional hybridization, their flanking regions typically remain unchanged. Transformation events may also contain unique junctions between segments of heterologous insert DNA and genomic DNA, or between two segments of genomic DNA, or between two segments of heterologous DNA. A “junction” is the point where two specific DNA fragments are joined. For example, junctions exist at the locations where insert DNA is joined to flanking DNA. Junction points also exist in transformed organisms where two DNA fragments are joined in a manner modified from that found in natural organisms. “Junctional DNA” refers to DNA containing the junction point.

The transgenic soybean event XP-2 of the present invention exhibits superior characteristics and performance compared to existing transgenic soybean plants and newly constructed events, comprising a DNA construct inserted in a single copy into the soybean genome. Said DNA construct (FIG. 1) comprises a T-DNA segment containing two linked plant expression cassettes: the CdP450 gene expression cassette and the cp4 epsps gene expression cassette. The DNA construct is introduced into the soybean genome using the Agrobacterium-mediated soybean cotyledonary node transformation method.

The present invention provides exemplary primers or probes that can be used to detect the presence in a sample of DNA originating from a soybean plant comprising Event XP-2 DNA. Such primers or probes are specific for target nucleic acid sequences and are thus suitable for identifying soybean Event XP-2 nucleic acid sequences by the methods of the invention.

The term “probe” refers to an isolated nucleic acid that is complementary to one strand of a target nucleic acid. Probes according to the invention not only include deoxyribonucleic acid or ribonucleic acid but also polyamides and other probe materials that specifically bind to a target DNA sequence and whose detection can be used to diagnose, differentiate, determine, or confirm the presence of the target DNA sequence in a particular sample. Probes may be linked to conventional detectable labels or reporter molecules, such as radioisotopes, ligands, chemiluminescent agents, or enzymes. An exemplary DNA molecule suitable for use as a probe is provided as SEQ ID NO:24.

The term “primer” may be a highly purified, isolated polynucleotide designed for use in specific annealing or hybridization methods involving thermal amplification. A pair of primers may be used together with template DNA, such as a sample of soybean genomic DNA, in thermal amplification such as polymerase chain reaction (PCR) to produce amplicons, wherein the amplicons produced by such reactions will have DNA sequences corresponding to the template DNA sequences located between the two sites where the primers hybridize to the template. As used herein, “amplicon” is a copy of a piece/fragment of DNA that has been synthesized using amplification techniques. Amplicons of the invention may comprise at least one sequence provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:10. Primers are typically designed to hybridize to a complementary target DNA strand to form a hybrid between the primer and the target DNA strand, and the presence of the primer is the point recognized by a polymerase to initiate extension of the primer using the target DNA strand as a template (i.e., additional nucleotides are polymerized into the elongating nucleotide molecule). As used in the invention, a pair of primers is intended to indicate the use of two primers that bind opposite strands of a double-stranded nucleotide segment, so as to linearly amplify, typically in a thermal amplification reaction or other conventional nucleic acid amplification method, a polynucleotide segment between the sites targeted for binding by the individual elements of the primer pair. Exemplary DNA molecules suitable for use as primers are provided as SEQ ID NO:22, SEQ ID NO:23, SEQ ID NO:25, or SEQ ID NO:26. The primer pair provided as SEQ ID NO:25 and SEQ ID NO:26 is suitable for use as a first DNA molecule and a second DNA molecule different from the first DNA molecule, both having a sufficient length of consecutive nucleotides of SEQ ID NO:10 to function as DNA primers that, when used in a thermal amplification reaction with template DNA originating from soybean Event XP-2, produce amplicons diagnostic for soybean Event XP-2 DNA in a sample.

Probes and primers according to the invention may have complete sequence identity with the target sequence, although primers and probes differing from the target sequence that retain the ability to preferentially hybridize to the target sequence may be designed by conventional methods. For a nucleic acid molecule to be useful as a primer or probe, it need only be sufficiently complementary in sequence to be able to form a stable double-stranded structure under the particular solvent and salt concentrations employed.

Any conventional nucleic acid hybridization or amplification method may be used to identify the presence of transgenic DNA from soybean Event XP-2 in a sample. Probes and primers are generally at least about 11, 18, 24, or 30 nucleotides or longer. Such probes and primers specifically hybridize to the target DNA sequence under stringent hybridization conditions. Conventional stringent conditions are those described by Sambrook et al., 1989, and by Haymes et al., in Nucleic Acid Hybridization, APractical Approach, IRL Press, Washington, DC (1985).

As used in the invention, “amplified DNA” or “amplicon” refers to a nucleic acid amplification product of a target nucleic acid sequence that is part of a nucleic acid template. For example, to determine whether a soybean plant was produced by sexual crossing with a plant containing transgenic soybean Event XP-2 of the invention, or whether a soybean sample taken from the field contains transgenic soybean Event XP-2, or whether a soybean extract, such as meal, flour, or oil, contains transgenic soybean Event XP-2, DNA extracted from a soybean plant tissue sample or extract may be subjected to a nucleic acid amplification method using a primer pair to produce amplicons diagnostic for the presence of transgenic soybean Event XP-2 DNA. The primer pair includes a first primer derived from a flanking sequence in the plant genome adjacent to the insertion site of the inserted exogenous DNA and a second primer derived from the inserted exogenous DNA. The amplicon has a length and sequence that are also diagnostic for the transgenic soybean Event XP-2. The length of the amplicon may range from the combined length of the primer pair plus one nucleotide base pair, preferably plus about fifty nucleotide base pairs, more preferably plus about two hundred fifty nucleotide base pairs, most preferably plus about four hundred fifty nucleotide base pairs or more.

Many methods well known to those skilled in the art may be used to isolate and manipulate the DNA molecules or fragments thereof disclosed in the invention, including thermal amplification methods. DNA molecules or fragments thereof may also be obtained by other techniques, such as by direct chemical synthesis of fragments, for example, using automated oligonucleotide synthesizers.

The term “progeny” or “offspring” includes any plant, seed, plant cell, and/or reproducible plant part comprising Event XP-2 DNA originating from an ancestral plant and/or comprising a DNA molecule having at least one sequence selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10. Plants, progeny, and seeds may be homozygous or heterozygous for the transgene. Progeny may be grown from seed produced by a plant containing soybean Event XP-2 and/or from seed produced by a plant fertilized with pollen from a plant containing soybean Event XP-2. Progeny plants may be produced by self-pollination (aka “selfing”) to produce true breeding plant lines, i.e., plants homozygous for the transgene. Appropriate selfing of progeny may produce plants homozygous for the incorporated exogenous gene. Alternatively, progeny plants may be outcrossed, for example, with another unrelated plant, to produce varieties or hybrid seeds or plants. The other unrelated plant may be transgenic or non-transgenic. Varieties or hybrid seeds or plants of the invention may thus be obtained by sexually crossing a first parent lacking the specific and unique DNA of soybean Event XP-2 with a second parent comprising soybean Event XP-2, thereby producing hybrids comprising the specific and unique DNA of soybean Event XP-2. Each parent may be a hybrid or inbred line/variety, provided that the crossing or breeding produces plants or seeds of the invention, i.e., having at least one allele comprising DNA of soybean Event XP-2 and/or a DNA molecule having at least one sequence selected from: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Two different transgenic plants may thus be crossed to produce hybrid progeny containing two independently segregated, incorporated, exogenous genes. For example, XP-2 conferring dual insect tolerance modes and glyphosate tolerance to soybean may be crossed with other transgenic soybean plants to produce plants having characteristics of both transgenic parents. An example would be the crossing of XP-2 conferring dual insect tolerance modes and glyphosate tolerance to soybean with a plant having one or more additional traits such as herbicide tolerance and/or pest control, resulting in progeny plants or seeds having dual tolerance modes to lepidopteran insect pests and at least one or more additional traits. Backcrossing with parental plants and outcrossing with non-transgenic plants, as well as asexual reproduction, may also be performed. Descriptions of other breeding methods commonly used for different traits and crops may be found in one of several references, such as Fehr, Breeding Methods for Cultivar Development, in Wilcox J. ed., American Society of Agronomy, Madison WI (1987).

The term “transgenic plant cell” is suitable for use in many industrial applications, including but not limited to: (i) use as a research tool for scientific inquiry or industrial research; (ii) use in cultures for producing endogenous or recombinant carbohydrate, lipid, nucleic acid, or protein products or small molecules, which may be used in subsequent scientific research or as industrial products; and (iii) use with modern plant tissue culture techniques to produce transgenic plants or plant tissue cultures, which may then be used in agricultural research or production. The production and use of microorganisms such as transgenic plant cells utilize modern microbiological techniques and human intervention to produce artificial, unique microorganisms. In this process, recombinant DNA is inserted into the genome of a plant cell to generate a transgenic plant cell that is distinct and unique from naturally occurring plant cells. This transgenic plant cell may then be cultured using modern microbiological techniques, similar to bacterial and yeast cells, and may exist in an undifferentiated unicellular state. The new genetic composition and phenotype of the transgenic plant cell are technical effects produced by integrating heterologous DNA into the cell's genome. Another aspect of the invention is a method of using the microorganisms of the invention. Methods of using the microorganisms of the invention, such as transgenic plant cells, include (i) methods of integrating the recombinant DNA into the genome of the cell to produce transgenic cells, then using the transgenic cells to obtain other cells with the same heterologous DNA; (ii) methods of culturing cells containing recombinant DNA using modern microbiological techniques; (iii) methods of producing and purifying endogenous or recombinant carbohydrate, lipid, nucleic acid, or protein products from cultured cells; and (iv) methods of producing transgenic plants or transgenic plant tissue cultures using modern plant tissue culture techniques with transgenic plant cells.

The term “commercial product” refers to any composition or product consisting of material derived from a soybean plant, whole or processed soybean seed, one or more plant cells, and/or plant parts comprising soybean Event XP-2 DNA. Commercial products may be sold to consumers and may be living or non-living. Non-living commercial products include, but are not limited to, non-living seed; whole or processed seeds, seed parts, and plant parts; soybean oil, soybean protein, soybean meal, soybean flour, soybean flakes, soybean hulls, soy milk, soy cheese, soy wine, animal feed comprising soybeans, paper comprising soybeans, cheese comprising soybeans, soybean biomass. and fuel products produced using soybean plants and soybean plant parts. Living commercial products include, but are not limited to, seeds, plants, and plant cells. Soybean plants comprising Event XP-2 may thus be used to manufacture any commercial product normally obtained from soybeans. Any such commercial product derived from a soybean plant comprising Event XP-2 may contain at least a detectable amount of DNA corresponding to the specific and unique DNA of soybean Event XP-2 and, in particular, may contain a detectable amount of a polynucleotide comprising a DNA molecule having at least one sequence selected from: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

Sequence Descriptions:
SEQ ID NO:1—A 26 bp nucleotide sequence adjacent to the 5′ transgene fragment insertion site and the junction with soybean genomic DNA in the transgenic soybean event XP-2.
SEQ ID NO:2—A 26 bp nucleotide sequence adjacent to the 3′ transgene fragment insertion site and the junction with soybean genomic DNA in the transgenic soybean event XP-2.
SEQ ID NO:3—A 60 bp nucleotide sequence adjacent to the 5′ transgene fragment insertion site and the junction with soybean genomic DNA in the transgenic soybean event XP-2.
SEQ ID NO:4—A 60 bp nucleotide sequence adjacent to the 3′ transgene fragment insertion site and the junction with soybean genomic DNA in the transgenic soybean event XP-2.
SEQ ID NO:5—A 100 bp nucleotide sequence adjacent to the 5′ transgene fragment insertion site and the junction with soybean genomic DNA in the transgenic soybean event XP-2.
SEQ ID NO:6—A 100 bp nucleotide sequence adjacent to the 3′ transgene fragment insertion site and the junction with soybean genomic DNA in the transgenic soybean event XP-2.
SEQ ID NO:7—A 1,747 bp nucleotide sequence adjacent to the 5′ transgene fragment insertion site and the junction with soybean genomic DNA in the transgenic soybean event XP-2.
SEQ ID NO:8—A 1,729 bp nucleotide sequence adjacent to the 3′ transgene fragment insertion site and the junction with soybean genomic DNA in the transgenic soybean event XP-2.
SEQ ID NO:9—The complete T-DNA insertion sequence.
SEQ ID NO:10—The entire T-DNA sequence, flanking soybean genomic sequences at both 5′ and 3′ ends.
SEQ ID NO:11—Primer 1 for fluorescence quantitative real-time PCR detection of the endogenous β-Lectin gene in soybean genome.
SEQ ID NO:12—Primer 2 for fluorescence quantitative real-time PCR detection of the endogenous, β-Lectin gene in soybean genome.
SEQ ID NO:13—Primer 1 for fluorescence quantitative real-time PCR screening of soybean transformation events.
SEQ ID NO:14—Primer 2 for fluorescence quantitative real-time PCR screening of soybean transformation events.
SEQ ID NO:15—Primer 1 for amplification of T-DNA flanking sequences.
SEQ ID NO:16—Primer 2 for amplification of T-DNA flanking sequences.
SEQ ID NO:17—Primer 3 for amplification of T-DNA flanking sequences.
SEQ ID NO: 18—Primer 4 for amplification of T-DNA flanking sequences.
SEQ ID NO:19—Primer 5 for amplification of T-DNA flanking sequences.
SEQ ID NO:20—Primer 6 for amplification of T-DNA flanking sequences.
SEQ ID NO:21—Primer 7 for amplification of T-DNA flanking sequences.
LAD1-1—Primer 8 for amplification of T-DNA flanking sequences.
LAD1-2—Primer 9 for amplification of T-DNA flanking sequences.
LAD1-3—Primer 10 for amplification of T-DNA flanking sequences.
LAD1-4—Primer 11 for amplification of T-DNA flanking sequences.
SEQ ID NO:22—Fluorescence quantitative real-time PCR primer SQ111 for identification of the transgenic soybean event XP-2. The sequence of oligonucleotide forward primer SQ111 (SEQ ID NO:22) is identical to the nucleotide sequences corresponding to positions 2612-2634 of SEQ ID NO:10 and positions 625-647 of SEQ ID NO:7.
SEQ ID NO:23—Fluorescence quantitative real-time PCR primer SQ112 for identification of the transgenic soybean event XP-2. The sequence of oligonucleotide reverse primer SQ112 (SEQ ID NO:23) is identical to the reverse complement nucleotide sequences corresponding to positions 2711-2739 of SEQ ID NO:10, positions 2-30 of SEQ ID NO:9, and positions 724-752 of SEQ ID NO:7.
SEQ ID NO:24—Probe PB113 for identification of the transgenic soybean event XP-2. The sequence of oligonucleotide probe PB113 (SEQ ID NO:24) is identical to the nucleotide sequences corresponding to positions 2678-2704 of SEQ ID NO: 10 and positions 691-717 of SEQ ID NO:7.
SEQ ID NO:25—PCR primer SQL 13 for detection of the transgenic soybean event XP-2. The sequence of oligonucleotide forward primer SQ114 (SEQ ID NO:25) is identical to the nucleotide sequences corresponding to positions 2549-2576 of SEQ ID NO:10 and positions 562-589 of SEQ ID NO:7.
SEQ ID NO:26—PCR primer SQ114 for detection of the transgenic soybean event XP-2. The sequence of oligonucleotide reverse primer SQ115 (SEQ ID NO:26) is identical to the reverse complement nucleotide sequences corresponding to positions 2768-2793 of SEQ ID NO:10, positions 59-84 of SEQ ID NO:9, and positions 781-806 of SEQ ID NO:7.
SEQ ID NO:27—Flanking soybean genomic sequence at the 5′ end of the T-DNA insertion sequence.
SEQ ID NO:28—Flanking soybean genomic sequence at the 3′ end of the T-DNA insertion sequence.

Compared with existing technologies, the beneficial effects of the present invention are mainly embodied in:
(1) The invention provides the transgenic soybean event XP-2 that exhibits tolerance to the phytotoxic effects of agricultural herbicides containing glyphosate and tolerance to the herbicide flazasulfuron. The genes encoding glyphosate and flazasulfuron tolerance traits are genetically linked within the same DNA segment and localized at a single locus in the genome of transgenic soybean event XP-2, which enhances breeding efficiency and enables molecular marker-assisted tracking of transgene insertion segments in breeding populations and their progeny. The transgenic soybean event XP-2 is deposited in the China Center for Type Culture Collection (CCTCC) as soybean (Glycine max) XP-2 seeds.
(2) The specific nucleic acid sequence provided by the invention for detecting soybean plants enable specific identification of transgenic soybean event XP-2. The sequence includes one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or their complementary sequences.
(3) The specific detection primer pairs or probes designed for the detection of the specific nucleic acid sequences (SEQ ID NO: 1-10 or their complementary sequences) can function as DNA primers or probes to generate amplification products diagnostic for transgenic soybean event XP-2 or its progeny, and can quickly, accurately and stably identify the presence of plant material derived from the transgenic soybean event XP-2. They can facilitate traceability and full-process supervision of XP-2 research, production, processing, and application.
(4) The progeny, agricultural products, or commodities derived from the transgenic soybean event XP-2 obtained through the methods of the invention have the characteristics of glyphosate and flazasulfuron tolerance.

### BRIEF DESCRIPTION OF THE DRAWINGS

FIG. 1. Schematic diagram of the transformation construct used to generate the transgenic soybean event.

FIG. 2. Structural schematic of exogenous inserted genes and the soybean genome.

FIG. 3. PCR results identifying tissues with any breeding activity containing transgenic soybean event XP-2; wherein M: DNA Marker; 1: Seed of transgenic soybean event XP-2; 2: Leaf of transgenic soybean event XP-2; 3: Flower pod of transgenic soybean event XP-2; 4: Negative control; 5: Non-transgenic soybean Wandou 28; 6: Transgenic soybean Zhonghuang 6106; 7: Conventional rice; 8: Conventional maize.

### DETAILED DESCRIPTION OF THE INVENTION

The present invention is further described below with specific examples. Those skilled in the art should understand that the techniques disclosed in the following examples represent methods found by the inventors to perform well in practicing the invention and may therefore be considered preferred embodiments for implementing the invention. However, it should be understood by those skilled in the art that many modifications can be made to the disclosed specific embodiments based on this disclosure, and similar or analogous results can still be obtained without departing from the spirit and scope of the invention. The scope of protection of the invention is not limited thereto:

The molecular biology and biochemical methods used in the following examples of the present invention are all known technologies. Detailed descriptions can be found in Current Protocols in Molecular Biology published by John Wiley and Sons, edited by Ausubel, and Molecular Cloning: A Laboratory Manual, 3rd Ed. published by Cold Spring Harbor Laboratory Press (2001), edited by J. Sambrook et al.

The specific formulations of the culture media used in the following examples of the present invention are as follows:
YEP Solid Medium Composition: Trvptone 10 g/L, Yeast extract 10 g/L, NaCl 5 g/L, agar 2.8 g/L, water as solvent, pH 7.0.
Germination Medium Composition: MS salts (Phytotech M524) 4.33 g/L, sucrose 20 g/L, agar 2.75 g/L, water as solvent, pH 5.8.
GADT Liquid Medium Composition: B5 salts (Phytotech G398) 0.32 g/L, MES 3.9 g/L, sucrose 30 g/L, water as solvent, pH 5.4. After autoclaving and cooling, add sterile-filtered 0.835 mg/L 6-benzylaminopurine (6-BA), 0.25 mg/L gibberellic acid A3 (GA3), 40 mg/L acetosyringone (AS), 154 mg/L DL-dithiothreitol (DTT), 1 mM sodium dithionite (S), and 2.4 g/L cysteine (Cys).
Rest Medium Composition: B5 salts (Phytotech G398) 3.21 g/L, MES 0.6 g/L, sucrose 30 g/L, agar 2.8 g/L, water as solvent, pH 5.7. After autoclaving and cooling, add sterile-filtered 0.835 mg/L 6-BA and 200 mg/L timentin.
Shoot Induction Medium Composition: B5 salts (Phytotech G398) 3.21 g/L, MES 0.6 g/L, sucrose 30 g/L, agar 2.8 g/L, water as solvent, pH 5.7. After autoclaving and cooling, add sterile-filtered 0.835 mg/L 6-BA, 200 mg/L timentin, and 25 mg/L glyphosate.
Shoot Elongation Medium Composition: MS salts and B5 vitamins mixture (Phytotech M404) 4.44 g/L, MES 0.59 g/L, asparagine 0.05 g/L, glutamine 0.05 g/L, sucrose 30 g/L, agar 2.8 g/L, water as solvent, pH 5.7. After autoclaving and cooling, add sterile-filtered 200 mg/L timentin, 25 mg/L glyphosate, 0.25 mg/L GA3, 1 mg/L zeatin, and 0.1 mg/L indole-3-acetic acid (IAA).
Rooting Medium Composition: MS salts and B5 vitamins mixture (Phytotech M404) 4.44 g/L, MES 0.59 g/L, sucrose 30 g/L, agar 2.8 g/L, water as solvent, pH 5.7. After autoclaving and cooling, add sterile-filtered 200 mg/L timentin, 25 mg/L glyphosate, and 0.1 mg/L IAA.

### Example 1: Generation of Transgenic Events

### (1) Acquisition of Plasmid Vectors Containing Exogenous Genes

The plasmid vector pXP used for soybean transfonnation in the present invention is illustrated in FIG. 1. The pXP vector was constructed by using pCambial300 (GenBank: AF234296.1) as the plant transformation backbone, and inserting a T-DNA containing a flazasulfuron-tolerant expression cassette (i.e., the complete CdP450 protein expression cassette) and a glyphosate-tolerant expression cassette (i.e., the cp4 epsps protein expression cassette) into the multiple cloning site region of the plant transformation vector backbone pCambia1300. The flazasulfuron-tolerant expression cassette: the promoter driving the CdP450 gene is the CsVMV promoter dcrived from Cassava vein mosaic virus, and the terminator is the NOS terminator from 35S of the Cauliflower mosaic virus (CaMV). The glyphosate-tolerate expression cassette: comprising an AtUbi promoter derived from Arabidopsis thaliana ubiquitin promoter, wherein said promoter drives the expression of cp4 epsps fused at its N-terminus to a rice CTP gene encoding signal peptide, and the terminator is the 35S gene terminator of the CaMV.

The T-DNA of plasmid vector pXP (SEQ ID NO:9, corresponding to positions 2710-8548 bp in SEQ ID NO:10) comprised the following elements and their positions were listed in Table 1: RB (right border) interval sequence (2710-2942, 233 bp), pCsVMV promoter (2943-3630, 688 bp), interval sequence (3631-3640, 10 bp), CdP450 (3641-5194, 1554 bp), interval sequence (5195-5200, 6 bp), CaMV35S terminator (5201-5391, 191 bp), interval sequence (5392-5409, 18 bp), pAtUbi promoter (5410-6673, 1264 bp), OsEPSPS CTP (6674-6895, 222 bp), cp4 epsps (6896-8263, 1368 bp), interval sequence (8264-8275, 12 bp), CaMV35S terminator (8276-8465, 190 bp), interval sequence (8466-8526, 61 bp), and LB (left border) interval sequence (8527-8548, 22 bp).

TABLE 1
genomes and genetic elements contained in SEQ ID NO: 10

| Position(length) | Name | Function |
|---|---|---|
| 1-2709 | Right flanking soybean | Flanking sequence of |
| (2709 bp) | genomic sequence | T-DNA right border in |
| | | soybean genome |
| 2710-2942 | RB(T-DNA right border) | Spacer sequence for vector |
| (233 bp) | interval sequence | construction |
| 2943-3630 | pCsVMV promoter | Initiating and regulating |
| (688 bp) | | CdP450 gene expression |
| 3631-3640 | interval sequence | Spacer sequence for vector |
| (10 bp) | | construction |
| 3641-5194 | CdP450 | Providing tolerance to |
| (1554 bp) | | flazasulfuron |
| 5195-5200 | interval sequence | Spacer sequence for vector |
| (6 bp) | | construction |
| 5201-5391 | CaMV35S terminator | Transcription termination |
| (191bp) | | signal |
| 5392-5409 | interval sequence | Spacer sequence for vector |
| (18 bp) | | construction |
| 5410-6673 | pAtUbi promoter | Driving cp4 epsps gene |
| (1264 bp) | | expression |
| 6674-6895 | OsEPSPS CTP | Rice EPSPS signal peptide |
| (222 bp) | | |
| 6896-8263 | cp4 epsps | Glyphosate-tolerant epsps |
| (1368 bp) | | gene |
| 8264-8275 | interval sequence | Spacer sequence for vector |
| (12 bp) | | construction |
| 8276-8465 | CaMV 35S terminator | Terminating cp4 epsps gene |
| (190 bp) | | expression |
| 8466-8526 | interval sequence | Spacer sequence for vector |
| (61 bp) | | construction |
| 8527-8548 | LB (left border) | T-DNA insertion into Left |
| (22 bp) | interval sequence | border region of the plant |
| | | genome |
| 8549-12718 | left flanking soybean | Flanking sequence of |
| (4170 bp) | genomic sequence | T-DNA left border in |
| | | soybean genome |

### (2) Agrobacterium Transformation

The plasmid vector pXP obtained in step (1) was introduced into Agrobacterium μLBA4404 via electroporation (2500 V) to generate Agrobacterium containing the transformed T-DNA vector.
(3) Soybean Genetic Transformation Soybean transfonnation followed the method reported by Li Guilan et al. (2005, “Research on Agrobacterium-mediated genetic transformation of soybean cotyledonary nodes,” Acta Agronomica Sinica, 31(2): 170-176), with glyphosate used as the screening compound. Specific steps were as follows:
\textcircled{1} Soybean seed sterilization (chlorine gas sterilization method): Mature soybean seeds of the Wandou 28 variety, selected for plumpness, absence of disease spots, cracks and hardness, were placed in 90×15 mm petri dishes (~150 seeds per dish in a single layer). Prior to sterilization, dishes were uncovered in a laminar flow hood with illumination and air circulation for 1 h, then transferred to a desiccator. A 250 mL beaker containing 30 mnL sodium hypochlorite solution and 70 mL water was placed in the desiccator, mixed thoroughly, and 8 mL of concentrated hydrochloric acid (36 wt %) was added, the desiccator was sealed immediately and left for ~12 h. Sterilized seeds were then transferred to a laminar flow hood for 1 h of air circulation to disperse residual chlorine gas.
\textcircled{2} Soybean seed germination: Sterilized seeds were inserted into Germination Medium (GM) with the hilum facing downward, submerging ~50% of each seed. Dishes containing 15 seeds each were incubated under light at 24° C. for 12 h to obtain swollen seeds.
\textcircled{3} Agrobacterium culture preparation: Agrobacterium containing the transformed vector from step (1) (stored at −80° C.) was streaked onto YEP solid medium and incubated in darkness at 24° C. for 12 h. A sterile 1 mL pipette tip was used to transfer bacteria to GADT liquid medium, vortexed, and adjusted to OD650=0.5 with additional GADT liquid medium to obtain the Agrobacterium suspension.
\textcircled{4} Explant preparation and Agrobacterium infection: the swollen seeds from step \textcircled{2} were placed on sterilized filter paper. A slanted cut was made with a #11 surgical blade to remove the tip of the embryonic root from the seed, and then the soybean seed was bisected along the axis. The seed coat was removed from the half cotyledon containing the embryo. Under a dissecting microscope, the two cotyledons were separated at the embryonic shoot, exposing the underlying meristem. The meristem was gently damaged with the surgical blade to obtain the explant. The prepared explants were immersed in the Agrobacterium culture from step \textcircled{3} for approximately 1.5 hours.
\textcircled{5} Co-cultivation: Excess Agrobacterium culture was removed, and inoculated explants were placed in petri dishes (15 explants per dish) and incubated in darkness at 26° C. for 3 d.
\textcircled{6} Recovery culture: Co-cultivated explants from step \textcircled{5} were inserted at a 300 angle into Rest Medium (RM), submerging ≈50% of each cotyledon. Dishes containing 7 explants each were incubated under a 16 h/8 h photoperiod at 26° C. with 3000 lx light intensity for 1 week.
\textcircled{7} Shoot Induction: Explants from step \textcircled{6} were transferred to Shoot Induction Medium (SIM) and arranged by the same method. Cultures were cultured under the same conditions (16 h/8 h photoperiod, 26° C., 3000 lx) for 3 weeks.
\textcircled{8} Shoot elongation: Explants with developed adventitious shoots were placed on sterilized filter paper. Cotyledons and yellowed parts were excised, and adventitious shoots were transferred to Shoot Elongation Medium (SEM) with basal portions submerged in medium (4-5 explants per dish). Cultures were maintained under the same conditions (16 h/8 h photoperiod, 26° C., 3000 lx) with medium replacement every 2 weeks until the shoots reached a length of approximately 3 cm.
\textcircled{9} Rooting: the shoots from step \textcircled{8} were cut off, and the cut surfaces were immersed in indole-3-butyric acid (IBA) solution for 2 min before transfer to Rooting Medium. Cultures were maintained under the same conditions (16 h/8 h photoperiod, 26° C., 3000 lx) for 1-2 weeks until the shoots have developed roots of approximately 2 cm in length, then the rooted plantlets were obtained.
\textcircled{10} Transplantation: the rooted plantlets from step \textcircled{9} were removed from medium, roots were rinsed with tap water to remove residual medium, yielding a total of 685 independent T₀ transgenic events (independent transformed plants).

### Example 2: Screening of Transgenic Soybean Events

After hardening, 685 T₀ generation transgenic events obtained from Example 1 were transplanted into natural soil in a greenhouse. Among them, 546 seedlings successfully survived in the greenhouse. When the T₀ transgenic soybean plants reached the V4 vegetative stage, 1800 g a.i/ha ofglyphosate was sprayed. Among them, 87 transformation events showed no phytotoxicity, 327 exhibited phytotoxicity, and 132 resulted in mortality (Table 2).

TABLE 2
Glyphosate Tolerance of TO Generation
Soybean Transgenic Events

| Glyphosate Dosage | Herbicide Response | Number of Events |
|---|---|---|
| 1800 g a.i./ha | no phytotoxicity | 87 |
| | Growth stunted, dwarfed | 327 |
| | plants | |
| | Plant death | 132 |

Quantitative PCR was performed on the transformation events without phytotoxicity to determine the content of exogenous genes in the 87 events, thereby assessing T-DNA insertion copy numbers. Transformation events carrying two or more copies were discarded. Plant genomic DNA was extracted from the aforementioned transformation events using the CTAB method. The copy number of the exogenous gene was determined by SYBR Green quantitative PCR. The Lectin gene from the soybean genome was selected as the reference gene, and a randomly chosen soybean transformation event was used as the baseline to calculate the relative concentration of the target gene at the beginning of the reaction.

This example utilized the SYBR Green qPCR kit (BIO RAD) for reactions conducted in a Bio-Rad CFX96TM Real-Time PCR instrument. Results were analyzed using the Ct value comparison method. The reaction system and protocols followed the instructions provided in the SYBR Green qPCR kit manual. Primer sequences were as follows:

TABLE 3
Primer Sequences for Quantitative PCR

| | Primer | |
|---|---|---|
| Sequence ID | Name | Primer Sequence (5′-3′) |
| SEQ ID NO: 11 | qLEC-FS | GCCCTCTACTCCACCCCCAT |
| SEQ ID NO: 12 | qLEC-RS | GCCCATCTGCAAGCCTTTTT |
| SEQ ID NO: 13 | XP-F | GAGCAGACCGCCATTCCCA |
| SEQ ID NO: 14 | XP-R | GAAGGCCATGCAGGCTATGG |

Through analysis of the experimental results regarding cp4 epsps gene copy numbers, it was confirmed that exogenous genes had integrated into the chromosomes of the tested soybean plants. Among these, there were 54 transgenic soybean events with a single copy integration.

A tolerance test using a higher dosage of flazasulfuron (112.5 g a.i./ha) was conducted on the progeny of the 54 selected single-copy transformation events. The results revealed that five transformation events demonstrated tolerance to the elevated flazasulfuron dosage: XP-2, XP-195, XP-325, XP-326, and XP-552 (Table 4).

TABLE 4
Glyphosate Tolerence of T₁ Generation Soybean Transgenic Events

| flazasulfuron dosage | Herbicide Response | Number of Events |
|---|---|---|
| 112.5 g a.i./ha | No phytotoxicity | 5 |
| | having phytotoxicity, New | 49 |
| | leaves getting yellow | |
| | Plant death | 0 |

The T₁ generation of five transformation events (XP-2, XP-195, XP-325, XP-326, and XP-552) were grown in a greenhouse. At the V4 stage, 225 g a.i./ha of flazasulfuron was sprayed, and the tolerance of each transformant was observed. The results revealed that XP-2 exhibited the highest tolerance to flazasulfuron.

Through evaluation of flazasulfuron tolerance, glyphosate tolerance, and field agronomic performance, transformation event XP-2 was ultimately selected as superior. This event demonstrated robust tolerance to both glyphosate and flazasulfuron, single-copy insertion of exogenous genes, excellent agronomic traits, and stable inheritance of these traits.

### Example 3: Detection of Soybean Transformation Event XP-2

### (1) Soybean Genome Extraction

Genomic DNA of soybean transformation event XP-2 was extracted using the CTAB (hexadecyltrimethylammonium bromide) method.

1000 mg of young leaves from the T₀ generation of soybean transformation event XP-2 were ground into powder in liquid nitrogen. Then, 0.8 mL of CTAB buffer (20 g/L CTAB, 1.4 M NaCl, 100 mM Tris-HCl, 20 mM EDTA, pH 8.0) preheated in a 65° C. water bath was added, and after thorough mixing, the mixture was incubated in the 65° C. water bath for 60 minutes.

An equal volume of chloroform was added, and the mixture was inverted to homogenize. After centrifugation at 12,000 rpm for 10 minutes, the supernatant was transferred to a new centrifuge tube.

0.7 volumes of isopropanol were added, and the tube was gently shaken before centrifugation at 12,000 rpm for 1 minute to pellet the DNA. The supernatant was discarded, and 1 mL of 75% ethanol was added to wash the pellet. Centrifugation at 12,000 rpm for 1 minute was repeated, followed by air-drying in a laminar flow hood.

The DNA pellet was dissolved in an appropriate volume of TE buffer (10 mM Tris-HCl, 1 mM EDTA, pH 8.0). DNA concentration was measured using a Nanodrop spectrophotomneter, and the samples were stored for subsequent use.

### (2) Analysis of Flanking DNA Sequences

The TAIL-PCR (Thermal asymmetric interlaced PCR) method reported by Liu et al. (Liu, Plant Journal 1995, 8(3): 457-463) was employed to determine the flanking genomic sequences at the exogenous gene insertion sites of the superior transformation event XP-2 screened in Example 1. Primer sequences are listed in Table 5, PCR reaction conditions in Table 6, and PCR reaction mixtures in Table 7.

TABLE 5
TAIL-PCR Primer Sequences

| SEQ ID | Primer | Sequence (5′-3′) |
|---|---|---|
| SEQ ID | LB-SP1 | TTTCTCCATAATAATGTGTGAGTAGTTCCC |
| NO: 15 | | |
| SEQID | LB-SP2a | ACGATGGACTCCAGTCCGGCCCTCATGTGTTGAGCATATAAGAA |
| NO: 16 | | ACCCTTAG |
| SEQ ID | LB-SP3 | CTAAAACCAAAATCCAGTACTAAAATCC |
| NO: 17 | | |
| SEQ ID | RB-0b | CGTGACTGGGAAAACCCTGGCGTT |
| NO: 18 | | |
| SEQ ID | RB-1b | ACGATGGACTCCAGTCCGGCCCAACTTAATCGCCTTGCAGCACA |
| NO: 19 | | TC |
| SEQ ID | RB-2b | GAAGAGGCCCGCACCGATCGCCCTT |
| NO: 20 | | |
| SEQ ID | AC1 | ACGATGGACTCCAGAG |
| NO: 21 | | |
| | LAD1-1 | ACGATGGACTCCAGAGCGGCCGCVNVNNNGGAA |
| | LAD1-2 | ACGATGGACTCCAGAGCGGCCGCBNBNNNGGTT |
| | LAD1-3 | ACGATGGACTCCAGAGCGGCCGCVVNVNNNCCAA |
| | LAD1-4 | ACGATGGACTCCAGAGCGGCCGCBDNBNNNCGGT |

In Table 5, V represents G, A, C, N represents A, T, C, G, and B represents G, T, C.

TABLE 6
TAIL-PCR Reaction Conditions

| Pre-amplification | | | Primary-amplification | | | Secondary-amplification | | |
|---|---|---|---|---|---|---|---|---|
| | Temperature | Time | | Temperature | Time | | Temperature | Time |
| Step | (° C.) | (min; s) | Step | (° C.) | (min; s) | Step | (° C.) | (min; s) |
| 1 | 93 | 2:00 | 1 | 94 | 0:20 | 1 | 94 | 0:20 |
| 2 | 95 | 1:00 | 2 | 65 | 1:00 | 2 | 68 | 1:00 |
| 3 | 94 | 0:30 | 3 | 72 | 3:00 | 3 | 72 | 3:00 |
| 4 | 60 | 1:00 | 4 | Go to step1 | 1 Time | 4 | 94 | 0:20 |
| 5 | 72 | 3:00 | 5 | 94 | 0:20 | 5 | 68 | 1:00 |
| 6 | Go to step3 | 10 Times | 6 | 68 | 1:00 | 6 | 72 | 3:00 |
| 7 | 94 | 0:30 | 7 | 72 | 3:00 | 7 | 94 | 0:20 |
| 8 | 25 | 2:00 | 8 | 94 | 0:20 | 8 | 50 | 1:00 |
| 9 | Ramping to72 | 0.5° C./S | 9 | 68 | 1:00 | 9 | 72 | 3:00 |
| 10 | 72 | 3:00 | 10 | 72 | 3:00 | 10 | Go to step1 | 6-7 Times |
| 11 | 94 | 0:20 | 11 | 94 | 0:20 | 11 | 72 | 5:00 |
| 12 | 58 | 1:00 | 12 | 50 | 1:00 | 12 | End | |
| 13 | 72 | 3:00 | 13 | 72 | 3:00 | 13 | | |
| 14 | Go to step11 | 25 Times | 14 | Go to step5 | 13 Times | 14 | | |
| 15 | 72 | 5:00 | 15 | 72 | 5:00 | 15 | | |
| 16 | End | | 16 | End | | 16 | | |

TABLE 7
PCR Reaction System

| Round1 | | | Round2 | | | Round3 | | |
|---|---|---|---|---|---|---|---|---|
| 2x Premix Ex | 10 | μL | 2x Premix Ex | 10 | μL | 2x Premix Ex | 20 | μL |
| Taq | | | Taq | | | Taq | | |
| LB-SP1/RB-0b | 0.5 | μL | LB-SP2a/RB-1b | 0.5 | μL | LB-SP3/RB-2b | 1 | μL |
| LAD mix | 2 | μL | AC1 | 0.5 | μL | AC1 | 1 | μL |
| Genomic DNA | 1 | μL | Round1 product | 1 | μL | Round1 product | 2 | μL |
| | | | 40-fold dilution | | | 10-fold dilution | | |
| ddH₂O | 6.5 | μL | ddH₂O | 8 | μL | ddH₂O | 16 | μL |

| | | |
|---|---|---|
| Total 20 μL | Total 20 μL | Total 40 μL |

The Round 2 or 3 PCR amplification products with strong specificity were recovered using the Axygen PCR product purification kit, cloned into the PMD20-T cloning vector (TaKaRa, Code: D107A), transformed into Escherichia coli, and the resulting positive clones were sequenced. The obtained sequence information was compared with the soybean online database (http://www.soybase.org) to search for similar soybean genomic sequences.

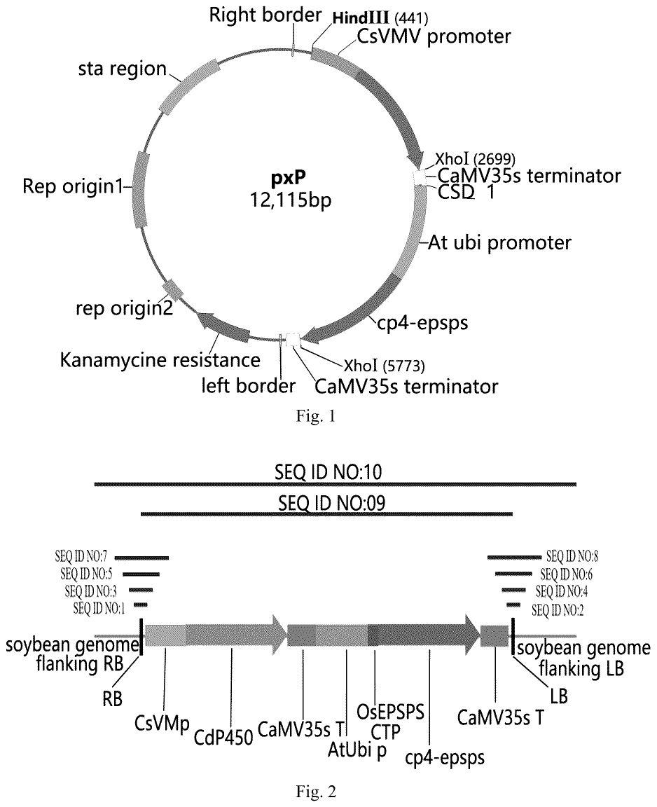

(3) Integration of XP-2 into Genomic Sequence Information
The aforementioned flanking sequences upstream and downstream of the confirmed insertion site (verified and validated by sequencing and alignment), the exogenous flazasulfuron-tolerant gene expression cassette, and the glyphosate-tolerant gene expression cassette were assembled to form the transformation event described in the present invention (schematic diagram shown in FIG. 2). The nucleotide sequence is provided as SEQ ID NO:10, and the genome and genetic elements contained in SEQ ID NO:10 are listed in Table 1. The corresponding soybean transformation event XP-2 was deposited in the China Center for Type Culture Collection in the form of XP-2 seeds of Glycine max μL. Merr., with the accession number CCTCC NO:P202329 and the date of deposit being Jul. 9, 2023.

### Example 4: Event-Specific Detection Primers and Probe for Transgenic Soybean Event XP-2

This example describes a method for identifying the presence of DNA from transgenic soybean event XP-2 in soybean samples. A pair of PCR primers and a probe were designed to detect the inserted T-DNA sequence of transgenic soybean event XP-2 and its right flanking soybean genomic sequence, with sequences encompassed within SEQ ID NO:1-10.

The PCR primers and probe used in this example were SQ111, SQ112, and PBI13. The oligonucleotide forward primer SQ111 (SEQ ID NO:22) was identical to the nucleotide sequence corresponding to positions 2612 to 2634 of SEQ ID NO: 10 and positions 625 to 647 of SEQ ID NO:7. The oligonucleotide reverse primer SQ112 (SEQ ID NO:23) was identical to the reverse complement of the nucleotide sequence corresponding to positions 2711 to 2739 of SEQ ID NO:10, positions 2 to 30 of SEQ ID NO:9, and positions 724 to 752 of SEQ ID NO:7. The oligonucleotide probe PB113 (SEQ ID NO:24) was identical to the nucleotide sequence corresponding to positions 2678 to 2704 of SEQ ID NO: 10 and positions 691 to 717 of SEQ ID NO:7. The PCR primers SQ111 (SEQ ID NO:22) and SQ112 (SEQ ID NO:23) amplified a 128-nucleotide amplicon unique to the genomic/inserted DNA junction in event XP-2. The probe PB113, labeled with a fluorescent dye (e.g., 6′-FAM), was used to detect the PCR products of primers SQ111 and SQ112, thereby identifying the presence of event XP-2-derived DNA in samples.

SEQ ID NO: 22:
gagagagaggcttctgtttccca;
SEQ ID NO: 23:
gccttcagtttaaactatcagtgtttgac;
SEQ ID NO: 24:
tcagattatgtgcagtgtctcgagcgc.

It should be apparent to those skilled in the art that, in addition to SQ111 (SEQ ID NO:22), SQ112 (SEQ ID NO:23), and PB113 (SEQ ID NO:24), other primers and/or probes could be designed to amplify and/or hybridize sequences within SEQ ID NO:10 that are used to detect the presence of event XP-2-derived DNA in a sample.

PCR assays for event identification were developed according to standard molecular biology laboratory practices to detect event XP-2 DNA in samples. Standard PCR assays or Parameters of PCR assays were optimized using the collections of primer pairs and probes (i.e., probes labeled with fluorescent tags such as 6FAMTM) designed to detect the presence of event XP-2-derived DNA in samples SQ111 (SEQ ID NO:22), SQ112 (SEQ ID NO:23), and PB113 (SEQ ID NO:24). Controls for the PCR reaction included internal control primers and internal control probes (e.g., VICTM-labeled) specific to a single-copy gene in the soybean genome. Those skilled in the art would know how to design primers specific to a single-copy gene in the soybean genome. Generally, parameters optimized for detecting event XP-2 DNA in samples included primer and probe concentrations, template DNA quantity, and PCR amplification cycling parameters.

### Example 5: Identification of Tissues with any Breeding Activity Containing Transgenic Soybean Event XP-2

This example utilized amplicons generated via PCR by a pair of primers to detect any tissues with breeding activity containing transgenic soybean event XP-2. The amplicon confirming the presence of transgenic soybean event XP-2 comprised at least 11 or more consecutive nucleotides provided in the form of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10. The primer pair included two primers, wherein one primer is located on the inserted expression cassette (SEQ ID NO: 9) and the other primer is located on the flanking sequence adjacent to the expression cassette.

To obtain diagnostic amplicons identifying SEQ ID NO: 1, SEQ ID NO:3, or SEQ ID NO:5 in this example, a forward primer molecule SQ114 (SEQ ID NO:25) was designed based on bases 1 to 1747 of SEQ ID NO:7, while a reverse primer molecule SQ115 (SEQ ID NO:26) was designed based on the inserted expression cassette DNA sequence (positions 1 to 8539 of SEQ ID NO:9), wherein the primer molecules had adjacent nucleotides of sufficient length to specifically hybridize to SEQ ID NO:7 and SEQ ID NO:9. The sequence of oligonucleotide forward primer SQ114 (SEQ ID NO:25) was identical to the nucleotide sequence corresponding to positions 2549 to 2576 of SEQ ID NO:10 and positions 562 to 589 of SEQ ID NO:7. The sequence of oligonucleotide reverse primer SQ115 (SEQ ID NO:26) was identical to the reverse complement of the nucleotide sequence corresponding to positions 2768 to 2793 of SEQ ID NO:10, positions 59 to 84 of SEQ ID NO:9, and positions 781 to 806 of SEQ ID NO:7.

SEQ ID NO: 25:
agggatagtgtttacattctctctagtc;
SEQ ID NO: 26:
gcctgaatggcgaatgctagagcagc.

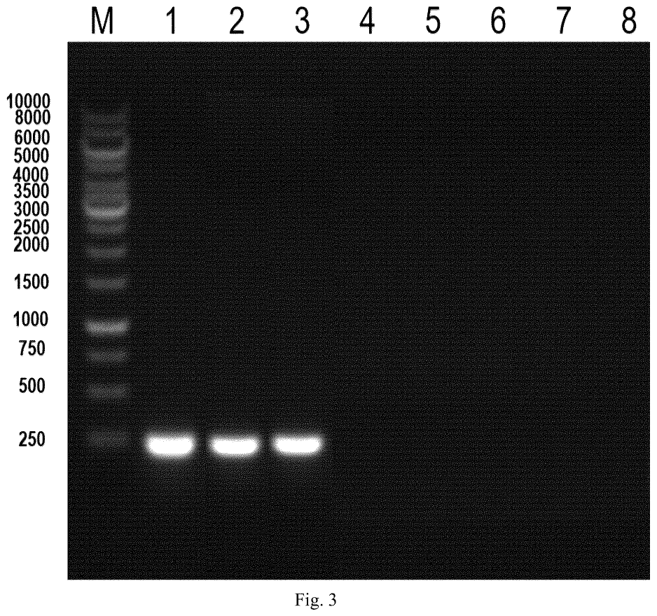

Genomic DNA was extracted from (1) seeds, (2) leaves, and (3) flower pods of transgenic soybean event XP-2 from Example 1 using the CTAB method and used as templates. PCR amplification was performed using primers SQ114 and SQ115 according to the reaction system outlined in Table 8, with the following controls: (4) a no-template control, (5) genomic DNA from non-transgenic soybean Wandou 28, (6) genomic DNA from transgenic soybean Zhonghuang 6106, (7) genomic DNA from conventional rice, and (8) genomic DNA from conventional maize. The amplification products were detected by agarose gel electrophoresis, and the results are shown in FIG. 3.

The PCR cycling conditions were as follows: denaturation at 95° C. for 3 min, denaturation at 95° C. for 15s, annealing at 58° C. for 30 s, and extension at 72° C. for 30 s. This cycle was repeated 32 times, followed by a final extension at 72° C. for 3 min.

TABLE 8
PCR Reaction System

| | | |
|---|---|---|
| Reagent | Final Concentration | Volume |

| | | | |
|---|---|---|---|
| TaKaRa LA Taq ® | 0.5× | 15 | μL |

| | | | | |
|---|---|---|---|---|
| 10 μmol/LForward Primer | 0.3 | μmol/L | 1.0 | μL |
| 10 μmol/Lreverse primer | 0.3 | μmol/L | 1.0 | μL |
| 200 mg/L DNA Template | 13.3 | mg/L | 2.0 | μL |

| | | | |
|---|---|---|---|
| ddH₂O | — | 11.0 | μL |
| Total Volume | — | 30.0 | μL |

Electrophoresis results of amplification products using primer pair SQ114 and SQ115 showed that only XP-2 samples yielded a detectable band of approximately 245 bp, which matched the expected size, while no specific bands were detected in other samples without the XP-2 genome. The primer pair provided in the present invention could specifically detect the presence of the XP-2 event (FIG. 3).

Apart from SQ114 (SEQ ID NO:25) and SQ115 (SEQ ID NO:26), it should be apparent to those skilled in the art that other primers could be designed to amplify sequences within SEQ ID NO: 10 that uniquely identify the presence of DNA derived from transgenic soybean event XP-2 in a sample and are used to detect the presence of such DNA. Alternative primer sequences could be selected by those skilled in DNA amplification methods from SEQ ID NO:7, SEQ ID NO:8, or SEQ ID NO:9. The use of such DNA primer sequences in modified versions of the methods described in this example falls within the scope of the present invention. The primer sequences of the invention that enable amplicon generation from XP-2-containing samples include at least one DNA primer sequence derived from SEQ ID NO:7., SEQ ID NO:8., or SEQ ID NO:9.

### Example 6: Protein Expression Quantification

Leaves (V4, V6, R2, R6), pods (R6), stems (R6), roots (R6), and seeds (R8) were collected from transgenic soybean event XP-2 across different generations (T4, T5, T6). The Cd P450 and cp4 epsps protein expression levels at various stages were detected using the Cd P450 Detection Kit (Enviro Logix Inc., USA) and the ep4 epsps ELISA Quantitative Detection Kit (Shanghai Yulong Biotechnology Co., Ltd.), respectively. Results are shown in Table 9. The data demonstrated stable hereditary expression of exogenous proteins in transgenic soybean event XP-2.

Cd P450 Detection Kit Procedure:
1. Sample and positive control preparation: Samples were ground in liquid nitrogen, and 20 mg of ground material was added to 1 mL of sample extraction buffer. The positive control provided in the kit was diluted with 2 mL of sample extraction buffer. After thorough mixing, samples were incubated on ice for 3 min, centrifuged at 12,000×g for 10 min, and the supernatant was diluted 20-200 times with PBS for loading.
2. Incubation: 50 μL of Cd P450 enzyme-linked reaction solution was added to each well of the ELISA plate. 50 μL of each sample and CdP450 protein standards (for standard curve construction) were added to respective wells. Plates were sealed with parafilm, incubated on a horizontal shaker at 180 rpm for 2 h at room temperature.
3. Washing: Plates were washed 3 times with 300 μL of washing buffer per well. After filling each plate, the buffer was discarded, and plates were inverted to remove residual liquid.
4. Color development: 100 μL of chromogenic substrate was added to each well, mixed thoroughly, and incubated at 180 rpm for 15-20 min at room temperature.
5. Termination: 100 μL of stop buffer was added to each well, mixed thoroughly, and absorbance was measured within 30 min.
6. Detection: Absorbance values of samples were analyzed at 450 nm using a Thermo MK3 microplate reader. A standard curve was generated using the positive control for target protein quantification.

cp4 epsps ELISA Quantitative Detection Kit Procedure:
1. Sample and positive control preparation: Samples were ground in liquid nitrogen, and 20 mg of ground material was added to 1 mL of sample extraction buffer. After thorough mixing, samples were incubated on ice for 3 min, centrifuged at 12,000×g for 10 min, and the supernatant was diluted 200-1000 times for loading.
2. Sample incubation: Diluted samples and cp4 epsps positive protein standards (for standard curve construction) were added to the ELISA plate (100 μL per well). Plates were incubated on a horizontal shaker at 180 rpm for 45 min at room temperature.
3. Washing: Plates were washed 3 times with 300 μL of washing buffer per well. After filling each plate, the buffer was discarded, and plates were inverted to remove residual liquid.
4. Enzyme-labeled antibody incubation: 100 μL of enzyme-labeled antibody was added to each well, and plates were incubated on a horizontal shaker at 180 rpm for 30 min at room temperature.
5. Washing: Plates were washed 3 times with 300 μL of washing buffer per well. After filling each plate, the buffer was discarded, and plates were inverted to remove residual liquid.
6. Color development: 100 μL of chromogenic substrate was added to each well, and plates were incubated at 180 rpm for 15-20 min at room temperature.
7. Termination: 100 μL of stop buffer was added to each well, mixed thoroughly, and absorbance was measured within 30 min.
8. Detection: Absorbance values of samples were analyzed at 450 nm using a Thermo MK3 microplate reader. A standard curve was generated using the positive control for target protein quantification.

TABLE 9
Expression levels of CdP450 and cp4 epsps
proteins in Transgenic Soybean Event XP-2

| | CdP450 | cp4 epsps |
|---|---|---|
| Soybean | μg/g fw ± SD* | μg/g fw ± SD* |
| Tissue | (Measurement Range) | (Measurement Range) |
| Leaf V4 | 2.24 ± 0.56 (1.38-3.04) | 62.7 ± 19.3 (38.1-95.2) |
| Leaf V6 | 2.39 ± 0.44 (1.69-3.20) | 63.2 ± 20.0 (33.3-94.8) |
| Leaf R6 | 2.77 ± 0.61 (1.61-3.81) | 138.8 ± 40.3 (78.7-182.4) |
| Stem R6 | 3.17 ± 0.40 (2.59-3.96) | 40.9 ± 12.2 (19.2-60.5) |
| Root R6 | 0.81 ± 0.23 (0.48-1.18) | 52.4 ± 21.2 (29.9-88.5) |
| Seed R8 | 136.65 ± 7.21 | 124.7 ± 8.4 (112.5-137.5) |
| | (118.81-144.76) | |

*Protein expression levels were reported in micrograms per gram fresh weight of plant tissue (μg/gfw). The values represent the mean ± standard deviation. n = 20 samples. The range in parentheses indicates the maximum and minimum values of the ELISA measurements.

### Example 7: Testing of Glyphosate, MCPA, Mesotrione, Penoxsulam, Nicosulfuron, Pyrazosulfuron-ethyl, and Flazasulfuron Tolerance in Transgenic Soybean Event XP-2

A randomized block design was adopted with a total of 24 plots, each measuring 4 m×5 m. Transgenic soybean event XP-2 and non-transgenic soybean variety Wandou 28 were double-seeded in each plot, maintaining a row spacing of 50 cm and plant spacing of 25 cm. A 1 m interval was set between adjacent plots, with three replicates for each soybean type. At the V3 growth stage, the following treatments were applied: 1) no herbicide application; 2) respective applications of glyphosate at 1800 g a.i./ha, MCPA at 1848 g a.i./ha, mesotrione at 123.75 g a.i./ha, penoxsulam at 45 g a.i./ha, nicosulfuron at 102 g a.i./ha, pyrazosulfuron-ethyl at 56.25 g a.i./ha, and flazasulfuron at 112.5 g a.i./ha. Seedling emergence rate, plant height (measured from the tallest 10 plants), and herbicide damage symptoms (evaluated from the 10 plants with the mildest symptoms) were investigated at 1, 2, and 4 weeks post-application. Herbicide damage symptom grading was performed according to GB/f 17980.42-2000. The formula for calculating herbicide damage rate was as follows:

$$X = \frac{\sum ( N \times S )}{T \times M} \times 100$$

(×-herbicide damage rate in percentage; N-number of plants with the same level of herbicide damage; S-herbicide damage level; T-total number of plants evaluated; M-highest level)_{∘}

Differences in emergence rate, seedling rate, and damage rate between transgenic soybean event XP-2 and non-transgenic soybean Wandou 28 across treatments were compared using analysis of variance (ANOVA). The tolerance level of transgenic soybean event XP-2 to herbicides was evaluated. Field test results indicated that transgenic soybean event XP-2 exhibited high tolerance to glyphosate, MCPA, mesotrione, penoxsulam, nicosulfuron, pyrazosulfuron-ethyl, and flazasulfuron (Table 10).

TABLE 10
Tolerance of Soybean Event XP-2 to Herbicides

| | Treatment | | | After 1 week | | After 2 weeks | | After 4 weeks | |
|---|---|---|---|---|---|---|---|---|---|
| | (effective | | Seedling | | damage | | damage | | damage |
| Herbicide | dose) | Material | rate % | Height(cm) | rate(%) | Height(cm) | rate(%) | Height(cm) | rate(%) |
| Glyphosate | No herbicide | Wandou 28 | 100 | 12.43 ± 0.44 | — | 14.75 ± 0.35 | — | 21.6 ± 0.65 | — |
| | application | XP-2 | 100 | 12.22 ± 0.34 | — | 14.74 ± 0.82 | — | 21.95 ± 0.97 | — |
| | 1800 g | Wandou 28 | 0 | — | 100 | — | 100 | — | 100 |
| | a.i./ha | XP-2 | 100 | 12.72 ± 1.01 | 0 | 14.58 ± 0.24 | 0 | 21.6 ± 0.52 | 0 |
| MCPA | No herbicide | Wandou 28 | 100 | 12.35 ± 0.4 | — | 14.85 ± 0.35 | — | 21.6 ± 0.65 | — |
| | application | XP-2 | 100 | 12.18 ± 0.28 | — | 14.79 ± 0.8 | — | 21.97 ± 0.96 | — |
| | 1848 g | Wandou 28 | 0 | — | 100 | — | 100 | — | 100 |
| | a.i./ha | XP-2 | 100 | 12.72 ± 1.01 | 0 | 14.7 ± 0.32 | 0 | 21.6 ± 0.52 | 0 |
| Mesotrione | No herbicide | Wandou 28 | 100 | 12.46 ± 0.43 | — | 14.76 ± 0.35 | — | 21.63 ± 0.65 | — |
| | application | XP-2 | 100 | 12.22 ± 0.34 | — | 14.75 ± 0.82 | — | 22 ± 0.95 | — |
| | 123.75 g | Wandou 28 | 0 | — | 100 | — | 100 | — | 100 |
| | a.i./ha | XP-2 | 100 | 12.76 ± 1.01 | 0 | 14.59 ± 0.24 | 0 | 21.63 ± 0.52 | 0 |
| Penoxsulam | No herbicide | Wandou 28 | 100 | 12.46 ± 0.44 | — | 14.77 ± 0.35 | — | 21.61 ± 0.65 | — |
| | application | XP-2 | 100 | 12.25 ± 0.34 | — | 14.76 ± 0.82 | — | 21.96 ± 0.97 | — |
| | 45 g a.i./ha | Wandou 28 | 0 | — | 100 | — | 100 | — | 100 |
| | | XP-2 | 100 | 12.75 ± 1.01 | 0 | 14.6 ± 0.24 | 0 | 21.61 ± 0.52 | 0 |
| Nicosulfuron | No herbicide | Wandou 28 | 100 | 12.49 ± 0.46 | — | 14.8 ± 0.22 | — | 21.63 ± 0.78 | — |
| | application | XP-2 | 100 | 12.27 ± 0.33 | — | 14.85 ± 0.75 | — | 21.97 ± 0.98 | — |
| | 102 g | Wandou 28 | 0 | — | 100 | — | 100 | — | 100 |
| | a.i./ha | XP-2 | 100 | 12.81 ± 1.01 | 0 | 14.73 ± 0.24 | 0 | 21.66 ± 0.53 | 0 |
| Pyrazosulfuron- | No herbicide | Wandou 28 | 100 | 12.4 ± 0.35 | — | 14.77 ± 0.35 | — | 21.63 ± 0.65 | — |
| ethyl | application | XP-2 | 100 | 12.39 ± 0.47 | — | 14.73 ± 0.79 | — | 21.98 ± 0.97 | — |
| | 56.25 g | Wandou 28 | 0 | — | 100 | — | 100 | — | 100 |
| | a.i./ha | XP-2 | 100 | 12.74 ± 1.3 | 0 | 14.6 ± 0.24 | 0 | 21.63 ± 0.52 | 0 |
| Flazasulfuron | No herbicide | Wandou 28 | 100 | 12.43 ± 0.44 | — | 14.75 ± 0.35 | — | 21.6 ± 0.65 | — |
| | application | XP-2 | 100 | 12.22 ± 0.34 | — | 14.74 ± 0.82 | — | 21.95 ± 0.97 | — |
| | 112.5 g | Wandou 28 | 0 | — | 100 | — | 100 | — | 100 |
| | a.i./ha | XP-2 | 100 | 12.49 ± 0.35 | 0 | 14.74 ± 0.2 | 0 | 21.69 ± 0.32 | 0 |

### Example 8: Testing of Tolerance in Transgenic Soybean Event XP-2 to Glyphosate+MCPA, Glyphosate+Mesotrione, Glyphosate+Penoxsulam, Glyphosate+Nicosulfuron, Glyphosate+Pyrazosulfuron-ethyl, and Glyphosate+Flazasulfuron

A randomized block design was employed with 24 plots, each measuring 4 mx5 m. Transgenic soybean event XP-2 and non-transgenic soybean Wandou 28 were double-seeded in each plot, maintaining a row spacing of 50 cm and plant spacing of 25 cm. A 1 m interval was set between adjacent plots, with four replicates for each soybean type. At the 3-5 leaf stage, the following treatments were applied: 1) no herbicide application; 2) respective applications of glyphosate at 900 g a.i./ha+MCPA at 337.5 g a.i./ha, glyphosate at 900 g a.i./ha+mesotrione at 123.75 g a.i./ha, glyphosate at 900 g a.i./ha+penoxsulam at 45 g a.i./ha, glyphosate at 900 g a.i./ha+nicosulfuron at 102 g a.i./ha, glyphosate at 900 g a.i./ha+pyrazosulfuron-ethyl at 5625 g a.i./ha, and glyphosate at 900 g a.i./ha+flazasulfuron at 112.5 g a.i./ha. Seedling rate, plant height (measured from the tallest 10 plants), and herbicide damage symptoms (evaluated from the 10 plants with the mildest symptoms) were investigated at 1, 2, and 4 weeks post-application. Herbicide damage symptom grading was performed according to GB/T 17980.42-2000. The formula for calculating herbicide damage rate was as follows:

$$X = \frac{\sum ( N \times S )}{T \times M} \times 100$$

(X-herbicide damage rate in percentage; N-number of plants with the same level of herbicide damage; S-herbicide damage level; T-total number of plants evaluated; M-highest level).

Differences in emergence rate, seedling rate, and damage rate between transgenic soybean event XP-2 and non-transgenic soybean Wandou 28 across treatments were compared using analysis of variance (ANOVA). The tolerance level of transgenic soybean event XP-2 to herbicide mixtures was evaluated. Field test results indicated that transgenic soybean event XP-2 exhibited high tolerance to glyphosate+MCPA, glyphosate+mesotrione, glyphosate+penoxsulam, glyphosate+nicosulfuron, glyphosate+pyrazosulfuron-ethyl, and glyphosate+flazasulfuron (Table 11).

TABLE 11
Investigation of Tolerance of Soybean Event XP-2 to Herbicide Mixtures

| | | | | | | | | | After 4 |
|---|---|---|---|---|---|---|---|---|---|
| | | | | After 1 week | | After 2 weeks | | | weeks |
| | Treatment | | Seedling | | damage | | damage | | damage |
| Herbicide | (effective dose) | Material | rate % | Height(cm) | rate(%) | Height(cm) | rate(%) | Height(cm) | rate(%) |
| Glyphosate + | No herbicide | Wandou 28 | 100 | 12.45 ± 0.44 | — | 14.79 ± 0.36 | — | 21.6 ± 0.65 | — |
| MCPA | application | XP-2 | 100 | 12.27 ± 0.33 | — | 14.78 ± 0.84 | — | 21.95 ± 0.97 | — |
| | 900 g a.i./ha + | Wandou 28 | 0 | — | 100 | — | 100 | — | 100 |
| | 337.5 g a.i./ha | XP-2 | 100 | 12.74 ± 1.01 | 0 | 14.63 ± 0.24 | 0 | 21.61 ± 0.52 | 0 |
| Glyphosate + | No herbicide | Wandou 28 | 100 | 12.46 ± 0.43 | — | 14.74 ± 0.36 | — | 21.6 ± 0.66 | — |
| Mesotrione | application | XP-2 | 100 | 12.25 ± 0.32 | — | 14.75 ± 0.84 | — | 21.96 ± 0.98 | — |
| | 900 g a.i./ha + | Wandou 28 | 0 | — | 100 | — | 100 | — | 100 |
| | 123.75 g a.i./ha | XP-2 | 100 | 12.77 ± 1.01 | 0 | 14.56 ± 0.26 | 0 | 21.62 ± 0.52 | 0 |
| Glyphosate + | No herbicide | Wandou 28 | 100 | 12.41 ± 0.44 | — | 14.75 ± 0.35 | — | 21.6 ± 0.66 | — |
| Penoxsulam | application | XP-2 | 100 | 12.23 ± 0.34 | — | 14.74 ± 0.85 | — | 21.98 ± 0.98 | — |
| | 900 g a.i./ha + | Wandou 28 | 0 | — | 100 | — | 100 | — | 100 |
| | 45 g a.i./ha | XP-2 | 100 | 12.72 ± 1.01 | 0 | 14.6 ± 0.24 | 0 | 21.64 ± 0.52 | 0 |
| Glyphosate + | No herbicide | Wandou 28 | 100 | 12.42 ± 0.43 | — | 14.73 ± 0.36 | — | 21.6 ± 0.65 | — |
| Nicosulfuron | application | XP-2 | 100 | 12.22 ± 0.34 | — | 14.73 ± 0.81 | — | 21.96 ± 0.98 | — |
| | 900 g a.i./ha + | Wandou 28 | 0 | — | 100 | — | 100 | — | 100 |
| | 102 g a.i./ha | XP-2 | 100 | 12.72 ± 1.01 | 0 | 14.56 ± 0.24 | 0 | 21.61 ± 0.52 | 0 |
| Glyphosate + | No herbicide | Wandou 28 | 100 | 12.45 ± 0.45 | — | 14.75 ± 0.35 | — | 21.59 ± 0.66 | — |
| Pyrazosulfuron- | application | XP-2 | 100 | 12.24 ± 0.34 | — | 14.75 ± 0.82 | — | 21.94 ± 0.96 | — |
| ethyl | 900 g a.i./ha + | Wandou 28 | 0 | — | 100 | — | 100 | — | 100 |
| | 56.25 g a.i./ha | XP-2 | 100 | 12.75 ± 1.01 | 0 | 14.59 ± 0.24 | 0 | 21.58 ± 0.52 | 0 |
| Glyphosate + | No herbicide | Wandou 28 | 100 | 12.46 ± 0.45 | — | 14.75 ± 0.35 | — | 21.6 ± 0.65 | — |
| Flazasulfuron | application | XP-2 | 100 | 12.25 ± 0.32 | — | 14.78 ± 0.82 | — | 21.95 ± 0.97 | — |
| | 900 g a.i./ha + | Wandou 28 | 0 | — | 100 | — | 100 | — | 100 |
| | 112.5 g a.i./ha | XP-2 | 100 | 12.77 ± 1.01 | 0 | 14.62 ± 0.24 | 0 | 21.62 ± 0.52 | 0 |

### Example 9: Hybridization to Generate Progeny Containing Transgenic Soybean Event XP-2

To produce soybean plants or plant parts with enhanced agronomic and herbicide-tolerant traits, soybean plants containing transgenic soybean event XP-2 were hybridized with soybean plants potentially containing any other soybean event or combination thereof, and phenotypes were evaluated to determine the resulting traits in progeny plants.

Specific operational steps for hybridization:
1. Flower Selection: On non-transgenic soybean plants (not event XP-2) that were disease/pest-free, undamaged, and healthy, flowers located at the upper-middle nodes (6th-12th node) were selected. Criteria included petals just beginning to emerge from the calyx, with petal color already visible.
2. Emasculation: The base of the flower peduncle and petals were gently pinched with the index finger and thumb of the left hand. The majority of the calyx was torn downward or diagonally downward using forceps held in the right hand, exposing the fused corolla. At this point, approximately the upper ⅓ of the corolla was clamped diagonally downward (at a ~45° angle relative to the peduncle) from the banner petal toward the keel petals (as the stigma curves toward the banner petal, this avoids damaging it), and gently lifted diagonally toward the banner petal.
3. Pollination: The sepals at the keel petals of the male parent flower (transgenic soybean event XP-2) were torn off. The corolla was split between the two keel petals to expose the yellow, fluffy-surfaced anthers. The entire pollen mass was then clamped from the middle of the filaments. The emasculated flower was gently held with the left hand, and the anthers were aligned with the stigma before lightly rubbing once or twice. The pollen mass was then carefully inverted onto the style to allow continued pollen dispersal and protect the exposed stigma.

The traits conferred on progeny plants by such plant breeding could extend beyond the herbicide tolerance of event XP-2, including but not limited to above-ground pest control, herbicide tolerance, nematocide properties, drought tolerance, virus resistance, antifungal control, bacterial resistance, male sterility, cold tolerance, salt tolerance, increased yield, enhanced oil composition, increased oil content, improved nutrient use efficiency, or altered amino acid content. Examples of transgenic events with improved agronomic traits are well-known in the art.

Below is a non-exhaustive list of potential transgenic soybean lines that may be developed through breeding from transgenic soybean event XP-2 to confer enhanced traits in soybean plants, plant parts, seeds, or commodity products. The breeding may incorporate any one or all combinations of the following: Herbicide Tolerance: Soybean GTS 40-3-2, MON87708, MON89788, A2704-12, A2704-21, A5547-35, A5547-127, BPS-CVl27-9, DP356043, GU262, W62, W98, DAS-44406-6, DAS-68416-4, FG72, BPS-CV127-9, SYHT04R, SYHTOH2, EE-GM3, pDAB4472-1606, pDAB4468-0416, pDAB8291.45.36.127, AAD-12; Insect Resistance: MON87701, DAS-81419-2; Enhanced Oil Composition: DP-305423, G94-1, G94-19, G168, OT96-15, MON87705, MON87769; Increased Yield: MON 87712.

1. A nucleic acid sequence of the transgenic soybean event XP-2, wherein the nucleic acid sequence comprises one or more selected from the group consisting of SEQ ID NO:1, SEQ ID NO:2, and their complementary sequences.

2. The nucleic acid sequence according to claim 1, wherein the nucleic acid sequence comprises one or more selected from the group consisting of SEQ ID NO:1-10 and their complementary sequences.

3. The nucleic acid sequence according to claim 1, wherein the nucleic acid sequence is derived from a plant, seed, or cell comprising the transgenic soybean event XP-2, and a representative sample of seeds comprising the transgenic soybean event XP-2 is deposited under Accession Number CCTCC NO: P202329.

4. A specific nucleotide sequence for detecting the transgenic soybean event XP-2, wherein the specific nucleotide sequence has at least 11 consecutive nucleotides of one of the nucleotide sequences shown in SEQ ID NO:1-10, or their complementary sequences.

5. A primer pair, wherein the primer pair comprises a first primer and a second primer different from the first primer, and the first primer and the second primer each comprise a portion of SEQ ID NO:10 or its complementary sequence for detecting the transgenic soybean event XP-2 in a sample.

6. The primer pair according to claim 5, wherein the first primer comprises at least 11 consecutive nucleotides of any segment of the transgenic region of SEQ ID NO:9 or its complementary sequence, and the second primer comprises a fragment of similar length from the 5′ and 3′ flanking soybean genomic DNA regions of SEQ ID NO:9 or their complementary sequences.

7. The primer pair according to claim 5, wherein the nucleotide sequence of the first primer is shown as SEQ ID NO: 22 or SEQ ID NO: 25; and the nucleotide sequence of the second primer is shown as SEQ ID NO: 23 or SEQ ID NO: 26.

8. A DNA probe, wherein the DNA probe comprises a portion of SEQ ID NO:10 or its complementary sequence, wherein the DNA probe hybridizes under stringent hybridization conditions to DNA containing any of SEQ ID NO:1-10 or their complementary sequences, but does not hybridize to DNA lacking these sequences.

9. The DNA probe according to claim 8, wherein the nucleotide sequence of the DNA probe is shown in SEQ ID NO:24.

10. A method for detecting the presence of DNA comprising the transgenic soybean event XP-2 in a sample, wherein the method comprises:
(1) contacting the sample to be tested with the DNA probe according to claim 8 in a nucleic acid amplification reaction mixture;
(2) performing an amplification reaction sufficient to generate DNA amplicons; and
(3) detecting the presence of DNA amplicons;
where the DNA amplicons comprise one of SEQ ID NO: 1-10 or their complementary sequences, the DNA comprising the transgenic soybean event XP-2 is present in the sample to be tested.

11. A method for cultivating herbicide-tolerant soybean plants containing the transgenic soybean event XP-2, wherein the method comprises: planting soybean seeds containing a specific nucleotide sequence, spraying a herbicide, and harvesting soybeans having significantly enhanced herbicide tolerance compared to other soybean plants lacking the specific nucleic acid sequence; wherein the specific nucleotide sequence is selected from one of SEQ ID NO:1-10, or their complementary sequences.

12. A method for controlling field weeds in the cultivation of soybean plants containing the transgenic soybean event XP-2, wherein the method comprises: planting transgenic soybean plants containing a specific nucleotide sequence, and spraying with effective doses of glyphosate and/or flazasulfuron herbicides to control the weeds; wherein the transgenic soybean genome comprises the specific nucleotide sequence from the transgenic soybean event XP-2, which is selected from one of SEQ ID NO:1-10, or their complementary sequences.

13. A method for obtaining flazasulfuron-tolerant and/or glyphosate-tolerant soybean plants based on the transgenic soybean event XP-2, wherein the method comprises: hybridizing the soybean plant containing a specific nucleotide sequence with another soybean plant to produce progeny plants; harvesting plants with significantly enhanced tolerance to herbicides compared to plants lacking the specific nucleotide sequence; wherein the specific nucleotide sequence is derived from the transgenic soybean event XP-2 and selected from one of SEQ ID NO:1-10, or their complementary sequences.

14. A soybean commodity or agricultural product produced from the transgenic soybean event XP-2, wherein the soybean commodity or agricultural product contains a detectable DNA molecule originating from the specific nucleotide sequence of the transgenic soybean event XP-2, which is selected from one of SEQ ID NO:1-10 or their complementary sequences; the soybean commodity or agricultural product is selected from the group consisting of: soybean oil, soybean protein, soybean meal, soybean flour, soybean flakes, soybean hulls, soymilk, soy cheese, soy wine, animal feed containing soybeans, paper containing soybeans, cheese containing soybeans, soybean biomass, and fuel products produced using soybean plants and soybean plant parts.

15. A method for detecting the presence of DNA comprising the transgenic soybean event XP-2 in a sample, wherein the method comprises:
(1) contacting the sample to be tested with the DNA probe according to claim 9 in a nucleic acid amplification reaction mixture;
(2) performing an amplification reaction sufficient to generate DNA amplicons; and
(3) detecting the presence of DNA amplicons;
where the DNA amplicons comprise one of SEQ ID NO: 1-10 or their complementary sequences, the DNA comprising the transgenic soybean event XP-2 is present in the sample to be tested.

16. A method for detecting the presence of DNA comprising the transgenic soybean event XP-2 in a sample, wherein the method comprises:
(1) contacting the sample to be tested with the primer pair according to claim 5 in a nucleic acid amplification reaction mixture;
(2) performing an amplification reaction sufficient to generate DNA amplicons; and
(3) detecting the presence of DNA amplicons;
where the DNA amplicons comprise one of SEQ ID NO: 1-10 or their complementary sequences, the DNA comprising the transgenic soybean event XP-2 is present in the sample to be tested.

17. A method for detecting the presence of DNA comprising the transgenic soybean event XP-2 in a sample, wherein the method comprises:
(1) contacting the sample to be tested with the primer pair according to claim 6 in a nucleic acid amplification reaction mixture;
(2) performing an amplification reaction sufficient to generate DNA amplicons; and
(3) detecting the presence of DNA amplicons;
where the DNA amplicons comprise one of SEQ ID NO: 1-10 or their complementary sequences, the DNA comprising the transgenic soybean event XP-2 is present in the sample to be tested.

18. A method for detecting the presence of DNA comprising the transgenic soybean event XP-2 in a sample, wherein the method comprises:
(1) contacting the sample to be tested with the primer pair according to claim 7 in a nucleic acid amplification reaction mixture;
(2) performing an amplification reaction sufficient to generate DNA amplicons; and
(3) detecting the presence of DNA amplicons;
where the DNA amplicons comprise one of SEQ ID NO: 1-10 or their complementary sequences, the DNA comprising the transgenic soybean event XP-2 is present in the sample to be tested.


SEQUENCE LISTING (28 sequences)

SEQ ID NO: 1 - DNA, 26 bp, synthetic construct
  source 1..26: mol_type = other DNA
cgagcgcaaaattgtcaaacactgat

SEQ ID NO: 2 - DNA, 25 bp, synthetic construct
  source 1..25: mol_type = other DNA
atacatctgttaaggatatattgtg

SEQ ID NO: 3 - DNA, 60 bp, synthetic construct
  source 1..60: mol_type = other DNA
attatgtgcagtgtctcgagcgcaaaattgtcaaacactgatagtttaaactgaaggcgg

SEQ ID NO: 4 - DNA, 60 bp, synthetic construct
  source 1..60: mol_type = other DNA
agcgtcaatttgtttacaccacaatatatccttaacagatgtatcacttgatgttttatt

SEQ ID NO: 5 - DNA, 100 bp, synthetic construct
  source 1..100: mol_type = other DNA
actgtagaaaatatttcagattatgtgcagtgtctcgagcgcaaaattgtcaaacactgatagtttaaactgaaggcgggaaacgacaatctgatccaag

SEQ ID NO: 6 - DNA, 100 bp, synthetic construct
  source 1..100: mol_type = other DNA
agagataaccaaaaaaatgaaataaaacatcaagtgatacatctgttaaggatatattgtggtgtaaacaaattgacgcttagacaacttaataacacat

SEQ ID NO: 7 - DNA, 1747 bp, synthetic construct
  source 1..1747: mol_type = other DNA
gtcatgtcatcaacaagggacagcttgccctgacaaggtaacagagaaagccaactgggttttggcagtttgctataggtggctgtaatgagaaagtcatttatgctgctgtttctaagttatgccatcattttcctcctttattttatattttcttttcatttttacactctttgtcttcttgaaatgggactggctttatttttcccttttttgttttccttccattagaggcagtttgtaataaccagtaaccactcttgtaattttgttgtcagggacataattctggattctaatatttagtgtttgaactttggagaggcatggaaacatcaagagagtaaatcagagggttgaaatcttcttcttttcattttttgtctattgtccaatattagaggatttggaagaaggaaaaatacttaaaatgaatagattgtacttactcaattatccttaattttgactacttaagtgaatctgaaacagttttaatacaaaaattgagaagagtttttattaataaaaaactcaatttcagaaattaattaatttatgagggatagtgtttacattctctctagtctctactgcaaaagtgcaaatagacattgacagggagagagagaggcttctgtttcccatgtttaatatacgcctattttaatcttaactgtagaaaatatttcagattatgtgcagtgtctcgagcgcaaaattgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttccagaaggtaattatccaagatgtagcatcaagaatccaatgtttacgggaaaaactatggaagtattatgtgaactcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcactagtgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtagatccaacaatggataaggcctacgtggccctcctctccttcgcctccctcttcttgctccactacctcgtttcccgccgcaatggcaccgggaagggcagca

SEQ ID NO: 8 - DNA, 1729 bp, synthetic construct
  source 1..1729: mol_type = other DNA
agcccatcatgaccagggatcataccgaaaagatgcttcaggggtttggcgccaacctgacggtcgagacggacgctgacggcgtcaggaccatccgccttgagggcaggggtaaactgactggccaagtcatcgatgttccgggagacccgtcgtccacggccttcccgttggttgcggcgctgctcgtgccggggagtgacgtgaccatcctgaacgtcctcatgaacccgaccaggaccggcctgatcctcacgcttcaggagatgggagccgacatcgaggtgatcaacccgcgcctggcaggcggtgaagacgttgcggatctgcgcgtgcgctcctctaccctgaagggcgtgacggtcccggaagatcgcgcgccgtccatgatagacgagtatcctattctggccgtcgccgctgcgttcgccgaaggggccacggtcatgaacggtcttgaggaactccgcgtgaaggaatcggatcgcctgtcggcggtggccaatggcctgaagctcaacggtgttgactgcgacgagggtgagacctcactcgtggtccgtggccggcctgatggcaagggcctcggcaacgccagtggagcggccgtcgccacgcacctcgatcatcgcatcgcgatgtccttcttggtgatgggtctcgtctcagagaacccggtgaccgtcgatgacgccacgatgatagcgacgagcttcccagagttcatggatctgatggcgggcctcggggccaagatcgaactgtctgacacgaaggccgcttgataggagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatccttaacagatgtatcacttgatgttttatttcatttttttggttatctctagtgtctttatattttaggaaccttgttaatttgaccctctatgaagacagaaaaaacagcttacctgactcctccttaaacatgactagcagcactatctgcaacaaagagtgaggcttcttctttctcaacaacaattccttcatatcttgtctttcttacctgtttgctggctctcttggtcaatggaatcttctcattatcattttcggtatgctcagtgaaatccaagccatgacttccctcactgtttatgcatacaaggtgaaggtgacttggaccagaagcagcatcagaagccatggaatcatcactctcttcatctgtatctcttttgaagtcatcattgttctctcttttgttgtcatcatcataaatgtttctatgagcattcttgtaattgttgccatagtcttcaaaatatacactgtgctcatcgtcaccattggagacttcattgtaggtaggagatccaatataagtggtccatccagactcagtgccattgaattccacttctgatgctaaatcttttgaacgatccatagtgaggcttttggcactggataacaaggaaaagcttagaagaaacaaaggaagcaaaagagga

SEQ ID NO: 9 - DNA, 5839 bp, synthetic construct
  source 1..5839: mol_type = other DNA
tgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttccagaaggtaattatccaagatgtagcatcaagaatccaatgtttacgggaaaaactatggaagtattatgtgaactcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcactagtgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtagatccaacaatggataaggcctacgtggccctcctctccttcgcctccctcttcttgctccactacctcgtttcccgccgcaatggcaccgggaagggcagcaaggccaagggcgcgctgccgccaagccctccatccgttccgttcctgggccacctccaccttgtcaagacgccattccacgctgcgctggcacgcctcgcggactgccacggcccggtcttctccctgcggatgggagcccgccccgcagttgtggtgtcctcgccggagcacgccaaggagtgcttcacggagcacgacgtggccttcgccaaccggccgcgctttccctcgcagcagctcgcctccttcaacggtgccgcgctgggttccgccagctacggcccgtactggcgcaacctccgccgcgtcgccaccgtccacctcctgtccgcgcaccgcgtcgcgtgcatgacggggactatcgcggccgaggtgcgggccatggtgcgacggatgaaccgcgccgcgcaggtggcatcaggcggcgcggcgcgcatcgagctcaagcggaggctatttgaggtctcgctcagcgtgcttatggagaccatcgcgcggaccaagacgtcacgtacggaggcggacgacgacacggacatgtcgcctgaggcccgggagttcaagcagatcgtggatgagctcctgcctcacctcggcacggctaacttgtgggactacatgccggtgttgcggtggttcgacgtgttcggcgtgaggaagaagatcgtgtccgcggtgaggagaagggacgcgttcctgcggcatcttgtcgacgcagagaggacgaggctggacgacggcaacgatgcgggcgagaagaagagcatcattgctatgctgctcactctgcagaagtcagagccggacgtctactcggataccatgatcatggctctatgtgggaacttgtttggggccggcacagagaccacgtcgacgaccaccgaatgggccatgtctctcctcctcaaccacccggagaagctcaggaaggcgcaggctgagatcgatgctgtcgtgggcacatcccgccttcttaccgccgacgacatgcctcgtctcacctacctccgctgcatcatcgacgagaccatgcgcctgtacccggccgcaccacttctgctgccacacgagtcctcgacacactgcaaggtcggcggctacgacgtgcccgccggcacgatgctgctcgtcaacgtgtacgccatccacagggaccccgcggtgtgggacgggccgaccgagttcgtgccggagcggttcgaggatggcaaggcagaaggccggctgctgatgccgttcgggatgggacggcgcaagtgtcccggcgagacgctcgcgctgcggacgatcgggctggtgctcggcacgctgatccagtgtttcgactgggaccgggttgatggtcttgaggtcgacatgactgaaagtggtgggctcacgatccccagggctgtcccgttggaggccatgtgcaggcctcgtgcgacgatgcgtgaggttttgcaggagctctgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaatttgaggtaccgagctcgaattctgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatggcggcgaccatggcgtccaacgctgcggctgcggctgcggtgtccctggaccaggccgtggctgcgtcggcagcgttctcgtcgcggaagcagctgcggctgcctgccgcagcgcgcggagggatgcgggtgcgggtgcgggcgcggggtcggcgggaggcggtggtggtggcgtccgcgtcgtcgtcgtcggtggcagcgccggcggcgaaggctgagatgctacacggtgcaagcagccggccggcaaccgctcgcaaatcttccggcctttcgggaacggtcaggattccgggcgataagtccatatcccaccggtcgttcatgttcggcggtcttgccagcggtgagacgcgcatcacgggcctgcttgaaggtgaggacgtgatcaataccgggaaggccatgcaggctatgggagcgcgtatccgcaaggaaggtgacacatggatcattgacggcgttgggaatggcggtctgctcgcccctgaggcccctctcgacttcggcaatgcggcgacgggctgcaggctcactatgggactggtcggggtgtacgacttcgatagcacgttcatcggagacgcctcgctcacaaagcgcccaatgggccgcgttctgaacccgttgcgcgagatgggcgtacaggtcaaatccgaggatggtgaccgtttgcccgttacgctgcgcgggccgaagacgcctaccccgattacctaccgcgtgccaatggcatccgcccaggtcaagtcagccgtgctcctcgccggactgaacactccgggcatcaccacggtgatcgagcccatcatgaccagggatcataccgaaaagatgcttcaggggtttggcgccaacctgacggtcgagacggacgctgacggcgtcaggaccatccgccttgagggcaggggtaaactgactggccaagtcatcgatgttccgggagacccgtcgtccacggccttcccgttggttgcggcgctgctcgtgccggggagtgacgtgaccatcctgaacgtcctcatgaacccgaccaggaccggcctgatcctcacgcttcaggagatgggagccgacatcgaggtgatcaacccgcgcctggcaggcggtgaagacgttgcggatctgcgcgtgcgctcctctaccctgaagggcgtgacggtcccggaagatcgcgcgccgtccatgatagacgagtatcctattctggccgtcgccgctgcgttcgccgaaggggccacggtcatgaacggtcttgaggaactccgcgtgaaggaatcggatcgcctgtcggcggtggccaatggcctgaagctcaacggtgttgactgcgacgagggtgagacctcactcgtggtccgtggccggcctgatggcaagggcctcggcaacgccagtggagcggccgtcgccacgcacctcgatcatcgcatcgcgatgtccttcttggtgatgggtctcgtctcagagaacccggtgaccgtcgatgacgccacgatgatagcgacgagcttcccagagttcatggatctgatggcgggcctcggggccaagatcgaactgtctgacacgaaggccgcttgataggagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcct

SEQ ID NO: 10 - DNA, 12718 bp, synthetic construct
  source 1..12718: mol_type = other DNA
cgacaatacactgctgcatctatcacatgataaaaaaaatcttattatgacaataaatataaaataaataatatacttcctcttgttcttattataataaggtatagttcaaaaaaattaattatcctttttataaagcactttataacttgtatgaaatatttaatttttttcattagttgcacttattaaatattttaagtatttttgtttttttattaataatggtgcataatactcttgaactttaatatattcatatccctcaaaacatgttaatgattatcaataagaacattattggtacattagttacatttttaaaaaattcaataaaattaaatgaattaattaattactattgacatgtgtgaatgatgtccaaagtaccttataataagaataagagagagtatataacactgacagtctaattgtatattgccattggaaattaaaatagtagagagtataagcaactctttgatttacagctcaggtggcaaaactgagtgctgaattgtgagtgtggtaacaatccatacaaaggaaaagagttgctaaactctcacatattgcagagatatataattgcacatattcatactttggtcaatgatacattccctagcataaggataccaccctaccccttttatttaagttaaacttagaaactcttaacagatgtatcactctatgaagacagaaaaaacagcttacctgactcctccttaaacatgactagcagcactatctgcaacaaagagtgaggcttcttctttctcaacaacaattccttcatatcttgtctttcttacctgtttgctggctctcttggtcaatggaatcttctcattatcattttcggtatgctcagtgaaatccaagccatgacttccctcactgtttatgcatacaaggtgaaggtgacttggaccagaagcagcatcagaagccatggaatcatcactctcttcatctgtatctcttttgaagtcatcattgttctctcttttgttgtcatcatcataaatgtttctatgagcattcttgtaattgttgccatagtcttcaaaatatacactgtgctcatcgtcaccattggagacttcattgtaggtaggagatccaatataagtggtccatccagactcagtgccattgaattccacttctgatgctaaatcttttgaacgatccatagtgaggcttttggcactggataacaaggaaaagcttagaagaaacaaaggaagcaaaagaggaaatgaactccaacaaatttggaacttgtgttttgctttctattctcagctgaaattccagtaatatttttataacctctcacccttcttccaatgctaacattcagttttacagataggacaaaaggcctatctcaacagtgcaggttcaactctcatttttgaattggcaatgtacctttggttacctggcaattctatctccatgagctcctttgatccaaccactagaagacaaggacttttggtaggaatacaataaaagtagaactaaaaagattgcactcaatctaaaaaaaaaatttagggtatctttgttggtgtgttcaattaaccttggctgaatgtagtcaacctttacttatatctgtacaatcttccatcaatagaatagagggtagtttttactaggaaaatgatacatgaacaaacatacctctattgtttgtacactccttgcatttttcaactttaaaatctttatcatatcatcaaaaggagttattatttataggtcataaaaaaaagtgaccatacagcaaaaaatattaaatagaaagggtccaacttaattctatatgaggaatgtgtgttacttaatagtgtctgcacatatcataacttctttcctcctattaaggttgagaacagatgaggagtgcccacataatttgttaatttaaacacataacttatcactttccacaccttcaaattcagtcatgtcatcaacaagggacagcttgccctgacaaggtaacagagaaagccaactgggttttggcagtttgctataggtggctgtaatgagaaagtcatttatgctgctgtttctaagttatgccatcattttcctcctttattttatattttcttttcatttttacactctttgtcttcttgaaatgggactggctttatttttcccttttttgttttccttccattagaggcagtttgtaataaccagtaaccactcttgtaattttgttgtcagggacataattctggattctaatatttagtgtttgaactttggagaggcatggaaacatcaagagagtaaatcagagggttgaaatcttcttcttttcattttttgtctattgtccaatattagaggatttggaagaaggaaaaatacttaaaatgaatagattgtacttactcaattatccttaattttgactacttaagtgaatctgaaacagttttaatacaaaaattgagaagagtttttattaataaaaaactcaatttcagaaattaattaatttatgagggatagtgtttacattctctctagtctctactgcaaaagtgcaaatagacattgacagggagagagagaggcttctgtttcccatgtttaatatacgcctattttaatcttaactgtagaaaatatttcagattatgtgcagtgtctcgagcgcaaaattgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttccagaaggtaattatccaagatgtagcatcaagaatccaatgtttacgggaaaaactatggaagtattatgtgaactcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcactagtgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtagatccaacaatggataaggcctacgtggccctcctctccttcgcctccctcttcttgctccactacctcgtttcccgccgcaatggcaccgggaagggcagcaaggccaagggcgcgctgccgccaagccctccatccgttccgttcctgggccacctccaccttgtcaagacgccattccacgctgcgctggcacgcctcgcggactgccacggcccggtcttctccctgcggatgggagcccgccccgcagttgtggtgtcctcgccggagcacgccaaggagtgcttcacggagcacgacgtggccttcgccaaccggccgcgctttccctcgcagcagctcgcctccttcaacggtgccgcgctgggttccgccagctacggcccgtactggcgcaacctccgccgcgtcgccaccgtccacctcctgtccgcgcaccgcgtcgcgtgcatgacggggactatcgcggccgaggtgcgggccatggtgcgacggatgaaccgcgccgcgcaggtggcatcaggcggcgcggcgcgcatcgagctcaagcggaggctatttgaggtctcgctcagcgtgcttatggagaccatcgcgcggaccaagacgtcacgtacggaggcggacgacgacacggacatgtcgcctgaggcccgggagttcaagcagatcgtggatgagctcctgcctcacctcggcacggctaacttgtgggactacatgccggtgttgcggtggttcgacgtgttcggcgtgaggaagaagatcgtgtccgcggtgaggagaagggacgcgttcctgcggcatcttgtcgacgcagagaggacgaggctggacgacggcaacgatgcgggcgagaagaagagcatcattgctatgctgctcactctgcagaagtcagagccggacgtctactcggataccatgatcatggctctatgtgggaacttgtttggggccggcacagagaccacgtcgacgaccaccgaatgggccatgtctctcctcctcaaccacccggagaagctcaggaaggcgcaggctgagatcgatgctgtcgtgggcacatcccgccttcttaccgccgacgacatgcctcgtctcacctacctccgctgcatcatcgacgagaccatgcgcctgtacccggccgcaccacttctgctgccacacgagtcctcgacacactgcaaggtcggcggctacgacgtgcccgccggcacgatgctgctcgtcaacgtgtacgccatccacagggaccccgcggtgtgggacgggccgaccgagttcgtgccggagcggttcgaggatggcaaggcagaaggccggctgctgatgccgttcgggatgggacggcgcaagtgtcccggcgagacgctcgcgctgcggacgatcgggctggtgctcggcacgctgatccagtgtttcgactgggaccgggttgatggtcttgaggtcgacatgactgaaagtggtgggctcacgatccccagggctgtcccgttggaggccatgtgcaggcctcgtgcgacgatgcgtgaggttttgcaggagctctgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaatttgaggtaccgagctcgaattctgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatggcggcgaccatggcgtccaacgctgcggctgcggctgcggtgtccctggaccaggccgtggctgcgtcggcagcgttctcgtcgcggaagcagctgcggctgcctgccgcagcgcgcggagggatgcgggtgcgggtgcgggcgcggggtcggcgggaggcggtggtggtggcgtccgcgtcgtcgtcgtcggtggcagcgccggcggcgaaggctgagatgctacacggtgcaagcagccggccggcaaccgctcgcaaatcttccggcctttcgggaacggtcaggattccgggcgataagtccatatcccaccggtcgttcatgttcggcggtcttgccagcggtgagacgcgcatcacgggcctgcttgaaggtgaggacgtgatcaataccgggaaggccatgcaggctatgggagcgcgtatccgcaaggaaggtgacacatggatcattgacggcgttgggaatggcggtctgctcgcccctgaggcccctctcgacttcggcaatgcggcgacgggctgcaggctcactatgggactggtcggggtgtacgacttcgatagcacgttcatcggagacgcctcgctcacaaagcgcccaatgggccgcgttctgaacccgttgcgcgagatgggcgtacaggtcaaatccgaggatggtgaccgtttgcccgttacgctgcgcgggccgaagacgcctaccccgattacctaccgcgtgccaatggcatccgcccaggtcaagtcagccgtgctcctcgccggactgaacactccgggcatcaccacggtgatcgagcccatcatgaccagggatcataccgaaaagatgcttcaggggtttggcgccaacctgacggtcgagacggacgctgacggcgtcaggaccatccgccttgagggcaggggtaaactgactggccaagtcatcgatgttccgggagacccgtcgtccacggccttcccgttggttgcggcgctgctcgtgccggggagtgacgtgaccatcctgaacgtcctcatgaacccgaccaggaccggcctgatcctcacgcttcaggagatgggagccgacatcgaggtgatcaacccgcgcctggcaggcggtgaagacgttgcggatctgcgcgtgcgctcctctaccctgaagggcgtgacggtcccggaagatcgcgcgccgtccatgatagacgagtatcctattctggccgtcgccgctgcgttcgccgaaggggccacggtcatgaacggtcttgaggaactccgcgtgaaggaatcggatcgcctgtcggcggtggccaatggcctgaagctcaacggtgttgactgcgacgagggtgagacctcactcgtggtccgtggccggcctgatggcaagggcctcggcaacgccagtggagcggccgtcgccacgcacctcgatcatcgcatcgcgatgtccttcttggtgatgggtctcgtctcagagaacccggtgaccgtcgatgacgccacgatgatagcgacgagcttcccagagttcatggatctgatggcgggcctcggggccaagatcgaactgtctgacacgaaggccgcttgataggagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatccttaacagatgtatcacttgatgttttatttcatttttttggttatctctagtgtctttatattttaggaaccttgttaatttgaccctctatgaagacagaaaaaacagcttacctgactcctccttaaacatgactagcagcactatctgcaacaaagagtgaggcttcttctttctcaacaacaattccttcatatcttgtctttcttacctgtttgctggctctcttggtcaatggaatcttctcattatcattttcggtatgctcagtgaaatccaagccatgacttccctcactgtttatgcatacaaggtgaaggtgacttggaccagaagcagcatcagaagccatggaatcatcactctcttcatctgtatctcttttgaagtcatcattgttctctcttttgttgtcatcatcataaatgtttctatgagcattcttgtaattgttgccatagtcttcaaaatatacactgtgctcatcgtcaccattggagacttcattgtaggtaggagatccaatataagtggtccatccagactcagtgccattgaattccacttctgatgctaaatcttttgaacgatccatagtgaggcttttggcactggataacaaggaaaagcttagaagaaacaaaggaagcaaaagaggaaatgaactccaacaaatttggaacttgtgttttgctttctattctcagctgaaattccagtaatatttttataacctctcacccttcttccaatgctaacattcagttttacagataggacaaaaggcctatctcaacagtgcaggttcaactctcatttttgaattggcaatgtacctttggttacctggcaattctatctccatgagctcctttgatccaaccactagaagacaaggacttttggtaggaatacaataaaagtagaactaaaaagattgcactcaatctaaaaaaaaaatttagggtatctttgttggtgtgttcaattaaccttggctgaatgtagtcaacctttacttatatctgtacaatcttccatcaatagaatagagggtagtttttactaggaaaatgatacatgaacaaacatacctctattgtttgtacactccttgcatttttcaactttaaaatctttatcatatcatcaaaaggagttattatttataggtcataaaaaaaagtgaccatacagcaaaaaatattaaatagaaagggtccaacttaattctatatgaggaatgtgtgttacttaatagtgtctgcacatatcataacttctttcctcctattaaggttgagaacagatgaggagtgcccacataatttgttaatttaaacacataacttatcactttccacaccttcaaattcagtcatgtcatcaacaagggacagcttgccctgacaaggtaacagagaaagccaactgggttttggcagtttgctataggtggctgtaatgagaaagtcatttatgctgctgtttctaagttatgccatcattttcctcctttattttatattttcttttcatttttacactctttgtcttcttgaaatgggactggctttatttttcccttttttgttttccttccattagaggcagtttgtaataaccagtaaccactcttgtaattttgttgtcagggacataattctggattctaatatttagtgtttgaactttggagaggcatggaaacatcaagagagtaaatcagagggttgaaatcttcttcttttcattttttgtctattgtccaatattagaggatttggaagaaggaaaaatacttaaaatgaatagattgtacttactcaattatccttaattttgactacttaagtgaatctgaaacagttttaatacaaaaattgagaagagtttttattaataaaaaactcaatttcagaaattaattaatttatgagggatagtgtttacattctctctagtctctactgcaaaagtgcaaatagacattgacagggagagagagaggcttctgtttcccatgtttaatatacgcctattttaatcttaactgtagaaaatatttcagattatgtgcagtgtctcgagcgcaaaattgtcaaacaagcttacagtaaatgatgttgatacgattaattaatttcaataaaatattagtctaattacacatttgattagttcgtaggagttataatgtagtcttgatgagaatgaaagaggaaaaagtcatgaattaatttgaatctttttcactaacaaaaggtaattatttaatttgcaataaagtcaaactttggagatataaattgcagttaagccaacatttataatgtagtcttgatagttaaagagagaatggaagagggagaaattatgaatttaaatcttttttattagtaagaactcacaaattaacaattaatattaattgataaaaaattgattaatttgcaataaagtcaaactttgaagataaaaattgcacttaagccaacatttagtaatagtaacattttttttaattctctctaattataaaaacattattgactaatatatgttcattagaaaattgattaatgtagttaatattattaaatttgtctataattatatattttttttaaaattactcttaagtaattaatgcatggataaataataatagtagtgaagttttaatttttaaagctaaaccctttcattattcaatcattataaaagagagaaagagaccggtcataacatttattgtttataaactaaattaattaagaatattttagtcaaaaaataattaatacaaggcacaagtaggaaaaaagtcttataagaaatttcaaaacaatttcttataaaaaaaaggaggtcggaagtagtaaaattaagggaaaattataaatttgttctttatctcagaattcaattttggttctcctataatttagtttatgaatttagtttttcattttttttaatcctattatttcagtctccaattccaaagttggatgtcaacttttacttgcttgctaacgttgaccgtcactggtcgtgcttttattggaagttggccattacatgtcgcatgttttttggatgttaattttgtgtacctaaattactttaaccttcaataaaatcgtgacacatgacggtcaacgtccaataaaaccgcaacgcgtgacggccaaggtaactaattaaccgtcaacatccaatttcagataacgggattgtaaaaaactgaagaattaaattcgtgaattaaattataaggagaccaaaatcgaattctgagataaataggaaaaccaaatttataatttagcgtaaaaattaaaaggagattaagtttttcaacatctaaataaactactatagaggcaaattattgttgcttcccactttgacagcatgatagcgaccattcgccaactcaatcttccgggaaagcattatttttcttgtttaactctatcatgttaccagaaagccttcaaaggtcatcagtttccttttggtgtttcagtgtttaattgtttcagtgtcttcttgcttgtcccttctcaataaaccaacacacaaagtagcacgtggatgggcaaatttacccaactgggtcagttttaaaaattaattatggaatggggtctcttttaaaagaatttgcactgggggtctgtttagtggaggggtatcgccattgaaaatggcagcaagcatggtatcgccagtgctgttggcgagacagcctggtaccgccagtgctgtttgcgagacaacctaggacagcctggttacgtggaaggtgccgccagtgagggtggcgagacacctccacgttggacccgcttcttctgatggcgatacaggttgcagacgtacagctttgttgcatgcatgctgaaaaagcagatacaggtgcacgttgcttcagcttttatattaatagggttgcaggttggtgcaacttatatttatagggttgcatgcatggtgaaaaagcagagtagagtaggcttaggtttttgaccgttggttctttgcattttttcttctataaaactaaccacaacatcttaaatttttacgcaagcttttcttctccaagctatcttctttctcagttattgagtttctgtgggtgagtgagagtgtggacgt

SEQ ID NO: 11 - DNA, 20 bp, synthetic construct
  source 1..20: mol_type = other DNA
gccctctactccacccccat

SEQ ID NO: 12 - DNA, 20 bp, synthetic construct
  source 1..20: mol_type = other DNA
gcccatctgcaagccttttt

SEQ ID NO: 13 - DNA, 19 bp, synthetic construct
  source 1..19: mol_type = other DNA
gagcagaccgccattccca

SEQ ID NO: 14 - DNA, 20 bp, synthetic construct
  source 1..20: mol_type = other DNA
gaaggccatgcaggctatgg

SEQ ID NO: 15 - DNA, 30 bp, synthetic construct
  source 1..30: mol_type = other DNA
tttctccataataatgtgtgagtagttccc

SEQ ID NO: 16 - DNA, 52 bp, synthetic construct
  source 1..52: mol_type = other DNA
acgatggactccagtccggccctcatgtgttgagcatataagaaacccttag

SEQ ID NO: 17 - DNA, 28 bp, synthetic construct
  source 1..28: mol_type = other DNA
ctaaaaccaaaatccagtactaaaatcc

SEQ ID NO: 18 - DNA, 24 bp, synthetic construct
  source 1..24: mol_type = other DNA
cgtgactgggaaaaccctggcgtt

SEQ ID NO: 19 - DNA, 46 bp, synthetic construct
  source 1..46: mol_type = other DNA
acgatggactccagtccggcccaacttaatcgccttgcagcacatc

SEQ ID NO: 20 - DNA, 25 bp, synthetic construct
  source 1..25: mol_type = other DNA
gaagaggcccgcaccgatcgccctt

SEQ ID NO: 21 - DNA, 16 bp, synthetic construct
  source 1..16: mol_type = other DNA
acgatggactccagag

SEQ ID NO: 22 - DNA, 23 bp, synthetic construct
  source 1..23: mol_type = other DNA
gagagagaggcttctgtttccca

SEQ ID NO: 23 - DNA, 29 bp, synthetic construct
  source 1..29: mol_type = other DNA
gccttcagtttaaactatcagtgtttgac

SEQ ID NO: 24 - DNA, 27 bp, synthetic construct
  source 1..27: mol_type = other DNA
tcagattatgtgcagtgtctcgagcgc

SEQ ID NO: 25 - DNA, 28 bp, synthetic construct
  source 1..28: mol_type = other DNA
agggatagtgtttacattctctctagtc

SEQ ID NO: 26 - DNA, 26 bp, synthetic construct
  source 1..26: mol_type = other DNA
gcctgaatggcgaatgctagagcagc

SEQ ID NO: 27 - DNA, 2709 bp, synthetic construct
  source 1..2709: mol_type = other DNA
cgacaatacactgctgcatctatcacatgataaaaaaaatcttattatgacaataaatataaaataaataatatacttcctcttgttcttattataataaggtatagttcaaaaaaattaattatcctttttataaagcactttataacttgtatgaaatatttaatttttttcattagttgcacttattaaatattttaagtatttttgtttttttattaataatggtgcataatactcttgaactttaatatattcatatccctcaaaacatgttaatgattatcaataagaacattattggtacattagttacatttttaaaaaattcaataaaattaaatgaattaattaattactattgacatgtgtgaatgatgtccaaagtaccttataataagaataagagagagtatataacactgacagtctaattgtatattgccattggaaattaaaatagtagagagtataagcaactctttgatttacagctcaggtggcaaaactgagtgctgaattgtgagtgtggtaacaatccatacaaaggaaaagagttgctaaactctcacatattgcagagatatataattgcacatattcatactttggtcaatgatacattccctagcataaggataccaccctaccccttttatttaagttaaacttagaaactcttaacagatgtatcactctatgaagacagaaaaaacagcttacctgactcctccttaaacatgactagcagcactatctgcaacaaagagtgaggcttcttctttctcaacaacaattccttcatatcttgtctttcttacctgtttgctggctctcttggtcaatggaatcttctcattatcattttcggtatgctcagtgaaatccaagccatgacttccctcactgtttatgcatacaaggtgaaggtgacttggaccagaagcagcatcagaagccatggaatcatcactctcttcatctgtatctcttttgaagtcatcattgttctctcttttgttgtcatcatcataaatgtttctatgagcattcttgtaattgttgccatagtcttcaaaatatacactgtgctcatcgtcaccattggagacttcattgtaggtaggagatccaatataagtggtccatccagactcagtgccattgaattccacttctgatgctaaatcttttgaacgatccatagtgaggcttttggcactggataacaaggaaaagcttagaagaaacaaaggaagcaaaagaggaaatgaactccaacaaatttggaacttgtgttttgctttctattctcagctgaaattccagtaatatttttataacctctcacccttcttccaatgctaacattcagttttacagataggacaaaaggcctatctcaacagtgcaggttcaactctcatttttgaattggcaatgtacctttggttacctggcaattctatctccatgagctcctttgatccaaccactagaagacaaggacttttggtaggaatacaataaaagtagaactaaaaagattgcactcaatctaaaaaaaaaatttagggtatctttgttggtgtgttcaattaaccttggctgaatgtagtcaacctttacttatatctgtacaatcttccatcaatagaatagagggtagtttttactaggaaaatgatacatgaacaaacatacctctattgtttgtacactccttgcatttttcaactttaaaatctttatcatatcatcaaaaggagttattatttataggtcataaaaaaaagtgaccatacagcaaaaaatattaaatagaaagggtccaacttaattctatatgaggaatgtgtgttacttaatagtgtctgcacatatcataacttctttcctcctattaaggttgagaacagatgaggagtgcccacataatttgttaatttaaacacataacttatcactttccacaccttcaaattcagtcatgtcatcaacaagggacagcttgccctgacaaggtaacagagaaagccaactgggttttggcagtttgctataggtggctgtaatgagaaagtcatttatgctgctgtttctaagttatgccatcattttcctcctttattttatattttcttttcatttttacactctttgtcttcttgaaatgggactggctttatttttcccttttttgttttccttccattagaggcagtttgtaataaccagtaaccactcttgtaattttgttgtcagggacataattctggattctaatatttagtgtttgaactttggagaggcatggaaacatcaagagagtaaatcagagggttgaaatcttcttcttttcattttttgtctattgtccaatattagaggatttggaagaaggaaaaatacttaaaatgaatagattgtacttactcaattatccttaattttgactacttaagtgaatctgaaacagttttaatacaaaaattgagaagagtttttattaataaaaaactcaatttcagaaattaattaatttatgagggatagtgtttacattctctctagtctctactgcaaaagtgcaaatagacattgacagggagagagagaggcttctgtttcccatgtttaatatacgcctattttaatcttaactgtagaaaatatttcagattatgtgcagtgtctcgagcgcaaaat

SEQ ID NO: 28 - DNA, 4170 bp, synthetic construct
  source 1..4170: mol_type = other DNA
taacagatgtatcacttgatgttttatttcatttttttggttatctctagtgtctttatattttaggaaccttgttaatttgaccctctatgaagacagaaaaaacagcttacctgactcctccttaaacatgactagcagcactatctgcaacaaagagtgaggcttcttctttctcaacaacaattccttcatatcttgtctttcttacctgtttgctggctctcttggtcaatggaatcttctcattatcattttcggtatgctcagtgaaatccaagccatgacttccctcactgtttatgcatacaaggtgaaggtgacttggaccagaagcagcatcagaagccatggaatcatcactctcttcatctgtatctcttttgaagtcatcattgttctctcttttgttgtcatcatcataaatgtttctatgagcattcttgtaattgttgccatagtcttcaaaatatacactgtgctcatcgtcaccattggagacttcattgtaggtaggagatccaatataagtggtccatccagactcagtgccattgaattccacttctgatgctaaatcttttgaacgatccatagtgaggcttttggcactggataacaaggaaaagcttagaagaaacaaaggaagcaaaagaggaaatgaactccaacaaatttggaacttgtgttttgctttctattctcagctgaaattccagtaatatttttataacctctcacccttcttccaatgctaacattcagttttacagataggacaaaaggcctatctcaacagtgcaggttcaactctcatttttgaattggcaatgtacctttggttacctggcaattctatctccatgagctcctttgatccaaccactagaagacaaggacttttggtaggaatacaataaaagtagaactaaaaagattgcactcaatctaaaaaaaaaatttagggtatctttgttggtgtgttcaattaaccttggctgaatgtagtcaacctttacttatatctgtacaatcttccatcaatagaatagagggtagtttttactaggaaaatgatacatgaacaaacatacctctattgtttgtacactccttgcatttttcaactttaaaatctttatcatatcatcaaaaggagttattatttataggtcataaaaaaaagtgaccatacagcaaaaaatattaaatagaaagggtccaacttaattctatatgaggaatgtgtgttacttaatagtgtctgcacatatcataacttctttcctcctattaaggttgagaacagatgaggagtgcccacataatttgttaatttaaacacataacttatcactttccacaccttcaaattcagtcatgtcatcaacaagggacagcttgccctgacaaggtaacagagaaagccaactgggttttggcagtttgctataggtggctgtaatgagaaagtcatttatgctgctgtttctaagttatgccatcattttcctcctttattttatattttcttttcatttttacactctttgtcttcttgaaatgggactggctttatttttcccttttttgttttccttccattagaggcagtttgtaataaccagtaaccactcttgtaattttgttgtcagggacataattctggattctaatatttagtgtttgaactttggagaggcatggaaacatcaagagagtaaatcagagggttgaaatcttcttcttttcattttttgtctattgtccaatattagaggatttggaagaaggaaaaatacttaaaatgaatagattgtacttactcaattatccttaattttgactacttaagtgaatctgaaacagttttaatacaaaaattgagaagagtttttattaataaaaaactcaatttcagaaattaattaatttatgagggatagtgtttacattctctctagtctctactgcaaaagtgcaaatagacattgacagggagagagagaggcttctgtttcccatgtttaatatacgcctattttaatcttaactgtagaaaatatttcagattatgtgcagtgtctcgagcgcaaaattgtcaaacaagcttacagtaaatgatgttgatacgattaattaatttcaataaaatattagtctaattacacatttgattagttcgtaggagttataatgtagtcttgatgagaatgaaagaggaaaaagtcatgaattaatttgaatctttttcactaacaaaaggtaattatttaatttgcaataaagtcaaactttggagatataaattgcagttaagccaacatttataatgtagtcttgatagttaaagagagaatggaagagggagaaattatgaatttaaatcttttttattagtaagaactcacaaattaacaattaatattaattgataaaaaattgattaatttgcaataaagtcaaactttgaagataaaaattgcacttaagccaacatttagtaatagtaacattttttttaattctctctaattataaaaacattattgactaatatatgttcattagaaaattgattaatgtagttaatattattaaatttgtctataattatatattttttttaaaattactcttaagtaattaatgcatggataaataataatagtagtgaagttttaatttttaaagctaaaccctttcattattcaatcattataaaagagagaaagagaccggtcataacatttattgtttataaactaaattaattaagaatattttagtcaaaaaataattaatacaaggcacaagtaggaaaaaagtcttataagaaatttcaaaacaatttcttataaaaaaaaggaggtcggaagtagtaaaattaagggaaaattataaatttgttctttatctcagaattcaattttggttctcctataatttagtttatgaatttagtttttcattttttttaatcctattatttcagtctccaattccaaagttggatgtcaacttttacttgcttgctaacgttgaccgtcactggtcgtgcttttattggaagttggccattacatgtcgcatgttttttggatgttaattttgtgtacctaaattactttaaccttcaataaaatcgtgacacatgacggtcaacgtccaataaaaccgcaacgcgtgacggccaaggtaactaattaaccgtcaacatccaatttcagataacgggattgtaaaaaactgaagaattaaattcgtgaattaaattataaggagaccaaaatcgaattctgagataaataggaaaaccaaatttataatttagcgtaaaaattaaaaggagattaagtttttcaacatctaaataaactactatagaggcaaattattgttgcttcccactttgacagcatgatagcgaccattcgccaactcaatcttccgggaaagcattatttttcttgtttaactctatcatgttaccagaaagccttcaaaggtcatcagtttccttttggtgtttcagtgtttaattgtttcagtgtcttcttgcttgtcccttctcaataaaccaacacacaaagtagcacgtggatgggcaaatttacccaactgggtcagttttaaaaattaattatggaatggggtctcttttaaaagaatttgcactgggggtctgtttagtggaggggtatcgccattgaaaatggcagcaagcatggtatcgccagtgctgttggcgagacagcctggtaccgccagtgctgtttgcgagacaacctaggacagcctggttacgtggaaggtgccgccagtgagggtggcgagacacctccacgttggacccgcttcttctgatggcgatacaggttgcagacgtacagctttgttgcatgcatgctgaaaaagcagatacaggtgcacgttgcttcagcttttatattaatagggttgcaggttggtgcaacttatatttatagggttgcatgcatggtgaaaaagcagagtagagtaggcttaggtttttgaccgttggttctttgcattttttcttctataaaactaaccacaacatcttaaatttttacgcaagcttttcttctccaagctatcttctttctcagttattgagtttctgtgggtgagtgagagtgtggacgt